(19) United States
(12) Patent Application Publication
(10) Pub. No.: US 2023/0094323 A1
(43) Pub. Date: Mar. 30, 2023

(54) System and method for optimizing general purpose biological network for drug response prediction using meta-reinforcement learning agent

(71) Applicant: KOREA ADVANCED INSTITUTE OF SCIENCE AND TECHNOLOGY, Daejeon (KR)

(72) Inventors: Kwang-Hyun CHO, Daejeon (KR); Yunseong KIM,, Daejeon (KR); Younghyun HAN, Daejeon (KR)

(21) Appl. No.: 17/898,755

(22) Filed: Aug. 30, 2022

(30) Foreign Application Priority Data
Sep. 15, 2021 (KR) 10-2021-0123488
May 20, 2022 (KR) 10-2022-0062120

(51) Int. Cl. G16C 20/70; G16C 20/30; G16H 20/10

(52) U.S. Cl. CPC G16C 20/70; G16C 20/30; G16H 20/10

(57) ABSTRACT

There is disclosed a method for determining a cancer treatment candidate drug including generating, by a simulation device, a plurality of specific perturbation networks by applying mutation information for a first cancer cell line to each of a plurality of drug responsive networks for a plurality of drugs, and selecting a plurality of candidate drugs from among the plurality of drugs based on a plurality of cell death probabilities for the first cancer cell line output by the plurality of specific perturbation networks.

### CROSS-REFERENCE TO RELATED APPLICATION

This application claims priority to Korean Patent Application No. 10-2021-0123488 filed on Sep. 15, 2021, and Korean Patent Application No. 10-2022-0062120 filed on May 20, 2022, and all the benefits accruing therefrom under 35 U.S.C. §119, the contents of which are incorporated by reference in their entirety.

### BACKGROUND

The present disclosure relates to relates to a technology for defining a biological network for predicting survival and death of cells by external stimuli to the cells, and implementing a method for optimizing parameters included in the biological network using a computing device, and implementing a method for determining a cancer treatment candidate drug using an in vitro test.

Cells can survive or die. Various proteins contained in a cell may contribute to the survival or death of the cell by influencing each other. Each of a set of proteins contained in a cell may affect the expression levels of other proteins according to its expression level. A meaningful network representing a relationship between proteins in one set may be constructed, and this may be referred to as a bio-signal transmission network or a biological network.

The biological network may be composed of nodes and links connecting between nodes. Each node may refer to a specific protein present in a cell. A weight may be assigned to each of the links. The weight may indicate the degree or strength of an influence of an expression level of a first protein, which represents a first node connected to a first end of both ends of the link corresponding to the weight, on an expression level of a second protein, which represents a second node connected to a second end of the both ends.

Various biological networks may be defined for one cell. Among them, one specific biological network may be particularly related to the expression and death of a specific cancer cell, and another biological network may be particularly related to the expression and death of another cancer cell.

When the cell is normal, a specific biological network defined for the cell may be referred to as a nominal biological network. The state of the cell may be determined by a combination of values of nodes of the nominal biological network. The value of each node may be determined by a state transition equation that determines time-dynamics of the nominal biological network. The state transition equation may depend on the weight of each link.

When the normal cell is transformed into a cancer cell due to a mutation in the cell, a certain node in the nominal biological network may not follow the state transition equation and may have a different type of time-dynamics. For example, a mutated node may always have only a specific value even after time passes. Such a mutated biological network may be referred to as a cancer cell biological network. The cancer cell biological network may have a feature that prevents the cancer cell from dying over time. In this case, when a specific drug is administered to the cancer cell, the specific drug may affect the expression level of a specific node of the cancer cell biological network, and cause the cancer cell to die by a chain action induced therefrom. In this case, it can be said that the specific drug perturbs the specific node. Furthermore, the specific drug may perturb one node or may perturb a plurality of nodes. Finding a drug that leads to death of the cancer cell may have a good effect in cancer treatment.

Actually, an optimal drug may be found through a test in which various drugs are sequentially administered to the cancer cell, but this method requires a lot of time and money, and in the meantime, the condition of a cancer patient may worsen and ultimately, treatment of the cancer patient may be unsuccessful. Therefore, when a simulation using a computing device may be used to quickly find a drug suitable for the cancer patient, it may be of great help in treating the cancer patient.

Some methods have been disclosed as such simulation methods.

Some of the previously disclosed technologies use the biological network described above. In this case, the reliability of the simulation result may be determined by the weight assigned to each link of the biological network. Therefore, it is important to find the optimal weights. The optimal weights may be determined by the experience and consideration of the researcher designing the simulation method on the biological network, but it is to be expected that there may be limitations.

Accordingly, the present disclosure is to provide a technique using machine learning to determine the optimal weights.

As the related art for a biological network and a method for determining a target drug using the same, Korean Patent Application Nos. 10-2109-0100505, 10-2018-0154390, 10-2107-0044192, 10-2017-0180959, 10-2013-0033843, etc. have been presented.

### SUMMARY

The present disclosure is to provide a technique for determining weights associated with links of a cancer cell biological network that may be commonly applied to various types of cancer and various patients regardless of the type of cancer and the location of a mutation.

The present disclosure is to provide a technique for optimizing parameters of a modeled biological network through machine learning to give the biological meaning of an internal structure thereof.

The present disclosure is to provide a technique for training an agent (a weight determining agent) that plays a role in determining weights assigned to the links in a biological network composed of nodes and links. In addition, the present disclosure is to provide a technique for selecting a drug suitable for the treatment of a new cancer patient using the agent for which training has been completed.

According to one aspect of the present disclosure, in a drug responsive network composed of nodes and links, an agent may be provided that is responsible for determining weights assigned to the links. When the weights assigned to the links of the drug responsive network are determined as appropriate values, a biological network may output a more accurate death probability of cells.

The agent may include a learnable network such as a machine learning network or a neural network, and may include a plurality of layers.

For training of the agent, a set of cancer cell lines for training, and a plurality of drugs (drug combination) may be used as training data. For one learning step, information about a set of cancer cell lines (N cancer cell lines) for training and one drug may be used.

When the drug is administered in vitro to the set of cancer cell lines for training, a percentage cell death may be determined through observation of the set of cancer cell lines for training. The percentage cell death may be presented, for example, as a vector Z composed of N scalar values.

Further, by applying the mutation information for the set of cancer cell lines for training to the drug responsive network, it is possible to generate a set of specific perturbation networks. Furthermore, it is possible to calculate a set of cell death probabilities that are obtainable from the set of specific perturbation networks. The percentage cell death may be presented, for example, as a vector Y composed of N scalar values.

A reward calculator provided according to an aspect of the present disclosure may calculate a reward that is a value to be input to the agent. The reward calculator may calculate the reward by using a distance between the vector Y and the vector Z.

The agent may receive the reward and weights assigned to the links of the drug responsive network as input data. The agent may output updated information for weights to be assigned to the links of the drug responsive network in the next learning step based on the input data.

The term ‘learning step’ in the present specification means updating the weights of the drug responsive network. In this regard, in order for the agent to be trained once, the learning step needs to be executed a plurality of times.

A set of a plurality of continuously executed learning steps may be referred to as a learning episode. For all learning steps in one episode, the drug used as training data may be limited to one. The drug for training may be changed after the episode has changed.

For one learning step, the values of the weights of the drug responsive network may be updated once. In addition, when the episode is executed once, the agent may be trained once. Each time the episode is repeated, the amount of training of the agent increases.

The agent, which has been sufficiently trained, may be used to select drugs for killing new cancer cell lines.

According to one aspect of the present disclosure, there is provided a computer-readable nonvolatile recording medium having a program thereon, the program instructions that cause a computing device to execute a learning step to decide weights of links of a drug responsive network responding to a specific drug, the learning step including: a first step of obtaining a cell death probability of a cancer cell line predicted by a specific perturbation network generated by applying mutation information for the cancer cell line to the drug responsive network, obtaining a percentage cell death of the cancer cell line obtained by performing an in vitro test of administering the specific drug to the cancer cell line, and calculating a reward value to which a difference value between the cell death probability and the percentage cell death is applied; a second step of calculating new weights for links in the drug responsive network by inputting the reward value to an agent; and a third step of updating the weights of the links of the drug responsive network with the new weights.

In this case, the program may further include instructions that cause the computing device to train the agent once based on a plurality of the rewards and a plurality of the new weights obtained in the process of executing the learning step a plurality of times.

In this case, in the first step, a step of obtaining the cell death probability [yp] of a cancer cell line [p] predicted by a p-th specific perturbation network generated by applying the mutation information for the cancer cell line [p], which is the p-th cancer cell line among N prepared cancer cell lines, to the drug responsive network, and obtaining the percentage cell death [zₚ] of the cancer cell line [p] obtained by performing the in vitro test of administering the specific drug to the cancer cell line [p] may be executed for each of the N cancer cell lines (p = 1, 2, 3, ... , N). Further, the first step may include calculating the reward value based on a first value that is a value inversely proportional to a distance between a vector Y composed of the cell death probabilities obtained for the N cancer cell lines and a vector Z composed of the percentage cell deaths obtained for the N cancer cell lines.

In this case, in the first step, a step of obtaining the cell death probability [yp] of a cancer cell line [p] predicted by a p-th specific perturbation network generated by applying the mutation information for the cancer cell line [p], which is the p-th cancer cell line among N prepared cancer cell lines, to the drug responsive network, and obtaining the percentage cell death [zₚ] of the cancer cell line [p] obtained by performing the in vitro test of administering the specific drug to the cancer cell line [p] may be executed for each of the N cancer cell lines (p = 1, 2, 3, ... , N). Further, the first step may include: calculating the first value that is a value inversely proportional to the distance between the vector Y composed of the cell death probabilities obtained for the N cancer cell lines and the vector Z composed of the percentage cell deaths obtained for the N cancer cell lines; and calculating the reward value based on a difference value between the first value and a second value prepared in advance. In this case, the second value may be a value inversely proportional to the distance between the past vector Y and the past vector Z obtained in the past learning step that has already been completed immediately before calculating the first value.

According to another aspect of the present disclosure, a computing device including a processing unit and a storage unit may be provided. The processing unit is configured to execute an episode, which is a process of training an agent to determine weights of links in a drug responsive network responding to a specific drug. The processing unit is configured to execute a predetermined learning step a plurality of times in executing the episode once. The learning step includes: a first step of obtaining a cell death probability of a cancer cell line predicted by a specific perturbation network generated by applying mutation information for the cancer cell line to the drug responsive network, obtaining a percentage cell death of the cancer cell line obtained by performing an in vitro test of administering the specific drug to the cancer cell line, and calculating a reward value to which a difference value between the cell death probability and the percentage cell death is applied; a second step of calculating new weights for links in the drug responsive network by inputting the reward value to the agent; and a third step of updating the weights of the links of the drug responsive network with the new weights.

In this case, in the first step, a step of obtaining the cell death probability [yp] of a cancer cell line [p] predicted by a p-th specific perturbation network generated by applying the mutation information for the cancer cell line [p], which is the p-th cancer cell line among N prepared cancer cell lines, to the drug responsive network, and obtaining the percentage cell death [zₚ] of the cancer cell line [p] obtained by performing the in vitro test of administering the specific drug to the cancer cell line [p] may be executed for each of the N cancer cell lines (p = 1, 2, 3, ... , N). The first step may include calculating the reward value based on a first value that is a value inversely proportional to a distance between a vector Y composed of the cell death probabilities obtained for the N cancer cell lines and a vector Z composed of the percentage cell deaths obtained for the N cancer cell lines.

In this case, the first step may include: calculating the first value that is a value inversely proportional to the distance between the vector Y composed of the cell death probabilities obtained for the N cancer cell lines and the vector Z composed of the percentage cell deaths obtained for the N cancer cell lines; and calculating the reward value based on a difference value between the first value and a second value prepared in advance. In this case, the second value may be a value inversely proportional to the distance between the past vector Y and the past vector Z obtained in the past learning step that has already been completed immediately before calculating the first value.

According to one aspect of the present disclosure, there is provided a method of generating a biological network including executing, by a computing device, a predetermined learning step. The learning step includes: a first step of the computing device obtaining a cell death probability of a cancer cell line predicted by a specific perturbation network generated by applying mutation information for the cancer cell line to a drug responsive network responding to a specific drug, obtaining a percentage cell death of the cancer cell line obtained by performing an in vitro test of administering the specific drug to the cancer cell line, and calculating a reward value to which a difference value between the cell death probability and the percentage cell death is applied; a second step of the computing device calculating new weights for links in the drug responsive network by inputting the reward value to the agent; and a third step of the computing device updating the weights of the links of the drug responsive network with the new weights.

In this case, the learning step may be repeatedly executed. Further, in the first step, a step of obtaining the cell death probability [yₚ] of a cancer cell line [p] predicted by a p-th specific perturbation network generated by applying the mutation information for the cancer cell line [p], which is the p-th cancer cell line among N prepared cancer cell lines, to the drug responsive network, and obtaining the percentage cell death [zₚ] of the cancer cell line [p] obtained by performing the in vitro test of administering the specific drug to the cancer cell line [p] may be executed for each of the N cancer cell lines (p = 1, 2, 3, ... , N). In this case, the first step may include: calculating the first value that is a value inversely proportional to the distance between the vector Y composed of the cell death probabilities obtained for the N cancer cell lines and the vector Z composed of the percentage cell deaths obtained for the N cancer cell lines; and calculating the reward value based on a difference value between the first value and a second value prepared in advance. In this case, the second value may be a value inversely proportional to the distance between the past vector Y and the past vector Z obtained in the past learning step that has already been completed immediately before calculating the first value.

According to one aspect of the present invention, a method for determining a cancer treatment candidate drug can be provided. The method comprises: generating, by a simulation device, a plurality of specific perturbation networks by applying mutation information for a first cancer cell line to each of a plurality of drug responsive networks for a plurality of drugs; selecting, by the simulation device, a plurality of candidate drugs from among the plurality of drugs based on a plurality of cell death probabilities for the first cancer cell line output by the plurality of specific perturbation networks; providing information on the plurality of determined candidate drugs to a drug response screening device; performing, by the drug response screening device, an in vitro test in which the plurality of candidate drugs are administered to a plurality of wells in which the first cancer cell line is stored; capturing, by the drug response screening device, images of the first cancer cell line in the plurality of wells using a cell image capturing device to analyze the captured images; and outputting, by the drug response screening device, a result of an in vitro test for at least some of the plurality of candidate drugs based on the analysis result.

The method may further comprises, prior to the generating, performing, by a computing device, a process (=episode) of determining weights of a k-th drug responsive network responding to a k-th drug among the plurality of drug responsive networks. In this case, in the performing of the process, an agent that has been trained by reinforcement learning may be used. And, in this case, the performing of the process may comprise: obtaining, by the computing device, mutation information for N (=pk) cell lines in which information on responsiveness by the in vitro test using the k-th drug among the plurality of drugs exists; generating, by the computing device, N specific perturbation networks by applying the N pieces of mutation information to the k-th drug responsive network responding to the k-th drug; repeatedly performing, by the computing device, a learning step a plurality of times by using the agent, the learning step being provided for updating the weights of the links of the k-th drug responsive network; observing, by the computing device, each reward provided to the agent at each learning step; selecting, by the computing device, a learning step corresponding to a reward with a largest value among the rewards observed at the observing step; and deciding, by the computing device, that the link weights output by the agent in the selected learning step are weights of the links of the k-th drug responsive network.

In this case, the agent may be configured to determine weights of the links of the k-th drug responsive network in a next learning step, based on the reward and the weights of the links of the k-th drug responsive network in a current learning step.

In this case, a process of determining the reward may comprise: preparing, by the computing device, a vector Y composed of N cell death probabilities output by the N specific perturbation networks and a vector z composed of N values related to a percentage cell death of the first cancer cell line observed by the in vitro test in which the k-th drug is administered to the first cancer cell line, in the current learning step in the plurality of times of the learning step; calculating, by the computing device, a first value inversely proportional to a distance between the vector Y and the vector Z; and calculating, by the computing device, the reward based on a difference value between the first value and a second value. In this case, the second value may be a value inversely proportional to a distance between the vector Y and the vector Z prepared in the learning step immediately before the current learning step.

The method may further comprises: training, by the computing device, the agent before the performing of the process (=episode) of determining the weights of the k-th drug responsive network. In this case, in the training of the agent, a process (=episode) of training the agent may be repeatedly performed for different G drugs. And, in this case, the process of training the agent that is performed for a g-th drug may comprise: obtaining, by the computing device, pg pieces of mutation information for cell lines in which information on responsiveness by the in vitro test using the g-th drug is present; generating, by the computing device, pg specific perturbation networks by applying the pg pieces of mutation information to a p-th drug responsive network responding to a p-th drug; repeatedly performing, by the computing device, a learning step a plurality of times by using the agent, the learning step being provided for updating the weights of the links of the g-th drug responsive network; and training, by the computing device, the agent by using the rewards provided to the agent during the plurality of learning steps and the weights obtained in a process of repeatedly performing the learning step a plurality of times.

In this case, the agent may be configured to determine weights of the links of the g-th drug responsive network in the next learning step, based on the reward and the weights of the links of the g-th drug responsive network in the current learning step.

According to another aspect of the present invention, a method for determining a cancer treatment candidate drug can be provided. The method comprises: performing, by a computing device, a process (=episode) of determining weights of a k-th drug responsive network responding to a k-th drug among a plurality of drug responsive networks for a plurality of drugs; generating, by a simulation device, a plurality of specific perturbation networks by applying mutation information for a first cancer cell line to each of the plurality of drug responsive networks; and selecting, by the simulation device, a plurality of candidate drugs from among the plurality of drugs based on a plurality of cell death probabilities for the first cancer cell line output by the plurality of specific perturbation networks. In this case, in the performing of the process, an agent that has been trained by reinforcement learning is used. In this case, the performing of the process comprises: obtaining, by the computing device, mutation information for N (=pk) cell lines in which information on responsiveness by the in vitro test using the k-th drug among the plurality of drugs exists; generating, by the computing device, N specific perturbation networks by applying the N pieces of mutation information to the k-th drug responsive network responding to the k-th drug; repeatedly performing, by the computing device, a learning step a plurality of times by using the agent, the learning step being provided for updating the weights of the links of the k-th drug responsive network; selecting, by the computing device, the learning step when a reward provided to the agent has a largest value among the plurality of times of the learning step; and deciding, by the computing device, that the link weights output by the agent in the selected learning step are weights of the links of the k-th drug responsive network.

According to still another aspect of the present invention, a system for determining a cancer treatment candidate drug can be provided. The system comprises: a simulation device; and a drug response screening device. In this case, the simulation device is configured to: generate a plurality of specific perturbation networks by applying mutation information for a first cancer cell line to each of a plurality of drug responsive networks for a plurality of drugs; select a plurality of candidate drugs from among the plurality of drugs based on a plurality of cell death probabilities for the first cancer cell line output by the plurality of specific perturbation networks; and provide information on the plurality of determined candidate drugs to a drug response screening device. In this case, the drug response screening device is configured to: perform an in vitro test in which the plurality of candidate drugs are administered to a plurality of wells in which the first cancer cell line is stored; capture images of the first cancer cell line in the plurality of wells using a cell image capturing device to analyze the captured images; and output a result of an in vitro test for at least some of the plurality of candidate drugs based on the analysis result.

The system may further comprise a computing device. In this case, the computing device may be configured to perform a process (=episode) of determining weights of a k-th drug responsive network responding to a k-th drug among the plurality of drug responsive networks before the simulation generates the plurality of specific perturbation networks. In this case, in performing the process, an agent that has been trained by reinforcement learning is used. In this case, the performing of the process may comprise: obtaining, by the computing device, mutation information for N (=pk) cell lines in which information on responsiveness by the in vitro test using the k-th drug among the plurality of drugs exists; generating, by the computing device, N specific perturbation networks by applying the N pieces of mutation information to the k-th drug responsive network responding to the k-th drug; repeatedly performing, by the computing device, a learning step a plurality of times by using the agent, the learning step being provided for updating the weights of the links of the k-th drug responsive network; selecting, by the computing device, the learning step when a reward provided to the agent has a largest value among the plurality of times of the learning step; and deciding, by the computing device, that the link weights output by the agent in the selected learning step are weights of the links of the k-th drug responsive network.

In this case, the agent may be configured to determine weights of the links of the k-th drug responsive network in a next learning step, based on the reward and the weights of the links of the k-th drug responsive network in a current learning step.

In this case, a process of determining the reward may comprise: preparing, by the computing device, a vector Y composed of N cell death probabilities output by the N specific perturbation networks and a vector z composed of N values related to a percentage cell death of the first cancer cell line observed by the in vitro test in which the k-th drug is administered to the first cancer cell line, in the current learning step in the plurality of times of the learning step; calculating, by the computing device, a first value inversely proportional to a distance between the vector Y and the vector Z; and calculating, by the computing device, the reward based on a difference value between the first value and a second value. In this case, the second value is a value inversely proportional to a distance between the vector Y and the vector Z prepared in the learning step immediately before the current learning step.

In this case, the computing device may be configured to train the agent before performing the process (=episode) of determining the weights of the k-th drug responsive network. In this case, in training the agent, a process (=episode) of training the agent may be repeatedly performed for different G drugs. In this case, the process of training the agent that is performed for a g-th drug may comprise: obtaining, by the computing device, pg pieces of mutation information for cell lines in which information on responsiveness by the in vitro test using the g-th drug is present; generating, by the computing device, pg specific perturbation networks by applying the pg pieces of mutation information to a p-th drug responsive network responding to a p-th drug; repeatedly performing, by the computing device, a learning step a plurality of times by using the agent, the learning step being provided for updating the weights of the links of the g-th drug responsive network; and training, by the computing device, the agent by using reward values and the weights obtained in a process of repeatedly performing the learning step a plurality of times.

In this case, the agent may be configured to determine weights of the links of the g-th drug responsive network in the next learning step, based on the reward and the weights of the links of the g-th drug responsive network in a current learning step.

According to still another aspect of the present invention, a system for determining a cancer treatment candidate drug can be provided. The system comprises: a simulation device; a drug response screening device; and a computing device. In this case, the computing device is configured to perform a process (=episode) of determining weights of a k-th drug responsive network responding to a k-th drug among a plurality of drug responsive networks. In this case, the simulation device is configured to: generate a plurality of specific perturbation networks by applying mutation information for a first cancer cell line to each of a plurality of drug responsive networks for a plurality of drugs; and select a plurality of candidate drugs from among the plurality of drugs based on a plurality of cell death probabilities for the first cancer cell line output by the plurality of specific perturbation networks. In this case, in the performing of the process, an agent that has been trained by reinforcement learning is used. In this case, the performing of the process comprises: obtaining, by the computing device, mutation information for N (=pk) cell lines in which information on responsiveness by the in vitro test using the k-th drug among the plurality of drugs exists; generating, by the computing device, N specific perturbation networks by applying the N pieces of mutation information to the k-th drug responsive network responding to the k-th drug; repeatedly performing, by the computing device, a learning step a plurality of times by using the agent, the learning step being provided for updating the weights of the links of the k-th drug responsive network; selecting, by the computing device, the learning step when a reward provided to the agent has a largest value among the plurality of times of the learning step; and deciding, by the computing device, that the link weights output by the agent in the selected learning step are weights of the links of the k-th drug responsive network.

### BRIEF DESCRIPTION OF THE DRAWINGS

Exemplary embodiments can be understood in more detail from the following description taken in conjunction with the accompanying drawings, in which:

FIG. 1a illustrates the concept of a biological network.

FIG. 1b is for describing the concept of a drug responsive network, which is a concept used in the present disclosure.

FIG. 1c shows the drug responsive network to which cell mutation information is applied.

FIG. 2a shows a method of defining and generating a plurality of specific perturbation networks different from each other from a specific drug responsive network according to an embodiment of the present disclosure.

FIG. 2b illustrates the method of generating the specific perturbation networks of FIG. 2a in another manner.

FIG. 3 shows a method of determining weights assigned to links of a specific drug responsive network according to an embodiment of the present disclosure.

FIG. 4 is a block diagram illustrating a function of the reward calculator calculating a reward value.

FIG. 5 is a block diagram illustrating a process of updating weights assigned to links of the nominal network using the calculated rewards.

FIG. 6 is a flowchart showing a method of updating weights assigned to links in a drug responsive network related to a specific drug by one learning step, which is provided by an embodiment of the present disclosure.

FIG. 7 shows a method of determining the weights of the drug responsive network as optimal values by using the weight update method of the drug responsive network described in FIG. 6.

FIG. 8 illustrates the concept of deciding a plurality of different drug responsive networks from a given nominal network.

FIG. 9 is a diagram illustrating a process of finding a drug suitable for a patient [x] using a plurality of decided different drug responsive networks, according to an embodiment of the present disclosure.

FIG. 10 shows a process of determining an optimal drug for a patient [k] using the K specific perturbation networks [x][k] prepared as in FIG. 9.

FIG. 11 shows a configuration of a computing device executing a method of completing a drug responsive network by determining weights of the drug responsive network according to an embodiment of the present disclosure.

FIG. 12 shows the configuration of a computing device executing a simulation method for determining an optimal drug effective for killing a specific cancer cell line according to an embodiment of the present disclosure.

FIG. 13 shows a structure of a cancer treatment candidate drug determination system provided according to an embodiment of the present disclosure.

FIG. 14a shows a framework of a k-th episode among K episodes for training the incomplete agent 20.

FIG. 14b shows a process of completing the training of the agent by executing a plurality of times of episodes according to an embodiment of the present disclosure.

FIG. 15 shows a configuration of a system for obtaining and providing a percentage cell death Z by administering a specific drug to cancer cell lines, according to an embodiment of the present disclosure.

FIG. 16a shows one example of a business model using the present disclosure.

FIG. 16b shows another example of a business model using the present disclosure.

### DETAILED DESCRIPTION OF EMBODIMENTS

Hereinafter, embodiments of the present disclosure will be described with reference to the drawings. However, the present disclosure is not limited to the embodiments described herein, and may be implemented by various modifications. The terms used herein are intended to aid understanding of the embodiments, and are not intended to limit the scope of the present disclosure. In addition, the singular forms used hereinafter include plural forms unless otherwise clearly expressed.

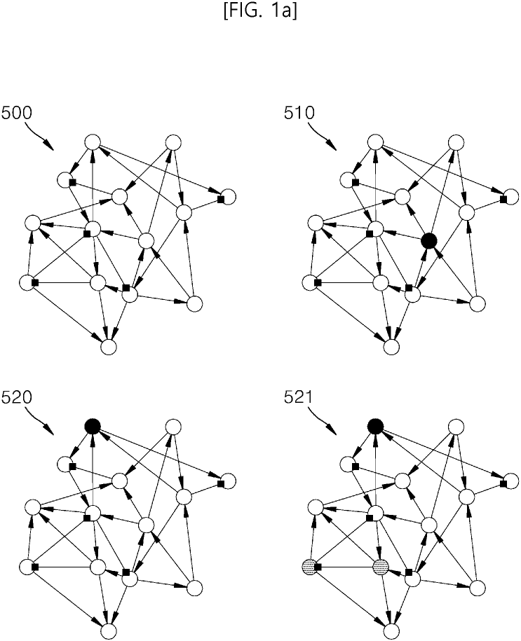

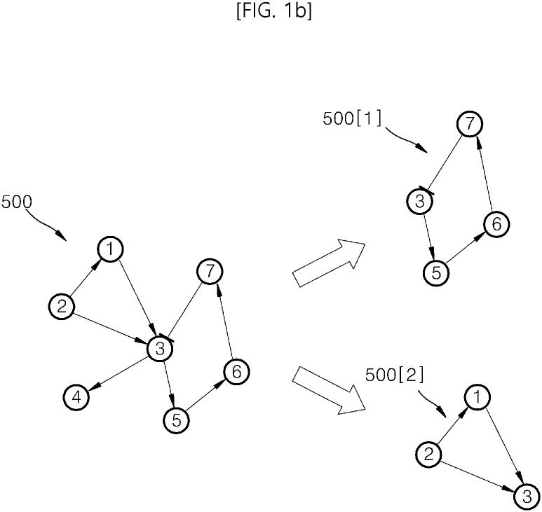

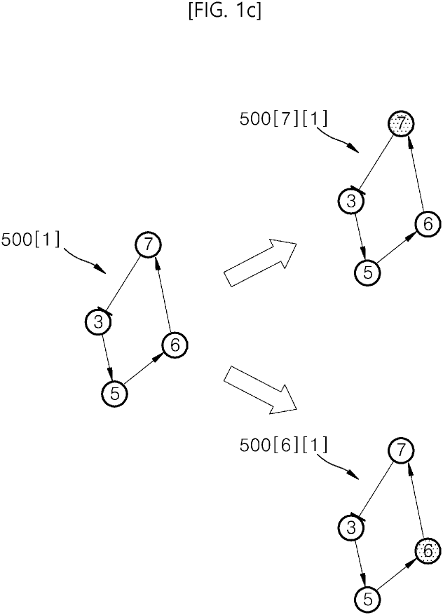

FIG. 1a illustrates the concept of a biological network.

In the present specification, a biological network may be referred to as a bio-signal transmission network, a biological signal transfer network, or a biological molecule network.

Reference number 500 conceptually suggests the structure of a specific biological network in a normal cell. What is indicated by reference number 500 may be referred to as a ‘nominal network’.

In an embodiment, the biological network may be composed of a plurality of nodes and a plurality of links connecting the nodes. In this case, each node represents the activity of a protein in the cell. Each node may be modeled to have a binary value or a real value. Each link represents the influence of the activity of a first node at the start point of a link on the activity of a second node at the endpoint (arrow or square) of the link. Links with their endpoints indicated by arrows indicate that the activity of the first node has a positive influence on the activity of the second node, and links with the endpoints marked with a rectangle indicate that the activity of the first node has a negative influence on the activity of the second node. A weight is assigned to each link, and the weight may indicate the strength of the positive or negative influence. The structure of the biological network may be constructed using knowledge revealed by the existing research in the field of biomolecules.

The modeling method may be selected from among a plurality of methods. Depending on different modeling methods, the number and expression methods of the types of links may be slightly different.

In the present specification, when a mutation exists in a specific node in the nominal network, the network in which the mutation exists may be referred to as a specific network. That is, the specific network may refer to a nominal network to which cell mutation information is applied.

Reference number 510 represents a ‘first specific network’, that is, ‘specific network [1]’, which indicates a case where a node corresponding to a mutation existing in a first cancer cell line, that is, the cancer cell line [1], exists in the nominal network. A node with mutation is marked in black.

Reference number 520 represents a ‘second specific network’, that is, ‘specific network [2]’, which indicates a case where a node corresponding to a mutation existing in a second cancer cell line, that is, the cancer cell line [2], exists in the nominal network. A node with mutation is marked in black.

The cancer cell line [k] may be described by replacing the concept and term of cancer cell [k].

As described above, when a node corresponding to a mutation in the cancer cell line [k] exists in the nominal network, it may be referred to as a ‘specific network [k]’ .

Reference number 521 represents a ‘specific perturbation network [2]’, which indicates a case where when a specific drug is administered to the cancer cell line [2], a target node that has the expression level affected by the specific drug exists in the specific network [2]. A node with a mutation is marked in black, and the two target nodes are marked in gray.

FIG. 1b is for describing the concept of a drug responsive network, which is a concept used in the present disclosure.

A plurality of different drug responsive networks may be defined from the nominal network 500. Each of the drug responsive networks may be regarded as a sub-network constituted by a part of the structure of the nominal network 500.

FIG. 1b shows a first drug responsive network 500[1] composed of nodes of node numbers 3, 5, 6, and 7 and a second drug responsive network 500[2] composed of nodes of node numbers 1, 2, and 3.

Although only two drug responsive networks defined from the nominal network 500 are shown in FIG. 1b, it can be easily understood that more drug responsive networks may be defined. For example, a k-th drug responsive network 500[k] not shown in FIG. 1b may be further defined.

In the k-th drug responsive network 500[k], state transition equations for determining a state value of each node at each time may have already been defined. The description of the state transition equations is exemplified in, for example, Korean Patent Nos. 10-2029297 and 10-1975424.

In this case, at least some of the coefficients included in the state transition equations may be determined by a weight assigned to each link of the k-th drug responsive network 500[k]. The weights have to be selected as optimal values. As a problem to be solved in the present disclosure, it is important to determine the optimal weight value to be assigned to each link of the k-th drug responsive network 500[k], and the means to solve the problem may be provided by specific embodiments of the present disclosure described below.

The different drug responsive networks are subnetworks having different substructures of the nominal network. Therefore, even if there is a link that exists in common in two different drug responsive networks, the weights assigned to the link may have different values for the two drug responsive networks.

In an embodiment of the present disclosure, a process of determining weights assigned to links existing in each drug responsive network among a plurality of drug responsive networks may be independently performed for each drug responsive network.

FIG. 1c shows the drug responsive network to which cell mutation information is applied.

When a mutation exists in node 7 of the nominal network 500, a drug responsive network 500[7][1] may be defined by applying the mutation information to the first drug responsive network 500[1] shown in FIG. 1b.

Alternatively, in the same way as above, when a mutation exists in node 6 of the nominal network 500, a drug responsive network 500[6][1] may be defined by applying the mutation information to the first drug responsive network 500[1] shown in FIG. 1b.

The drug responsive network obtained by applying the cell mutation information in this way may be referred to as a specific perturbation network.

FIG. 1c exemplifies two specific perturbation networks defined from the first drug responsive network 500[1], but it could be easily understood that other mutation information is used to define a larger number of specific perturbation networks.

The aforementioned FIGS. 1a, 1b, and 1c may be collectively referred to as FIG. 1.

### Process of Deciding Weights of Specific Drug Responsive Network

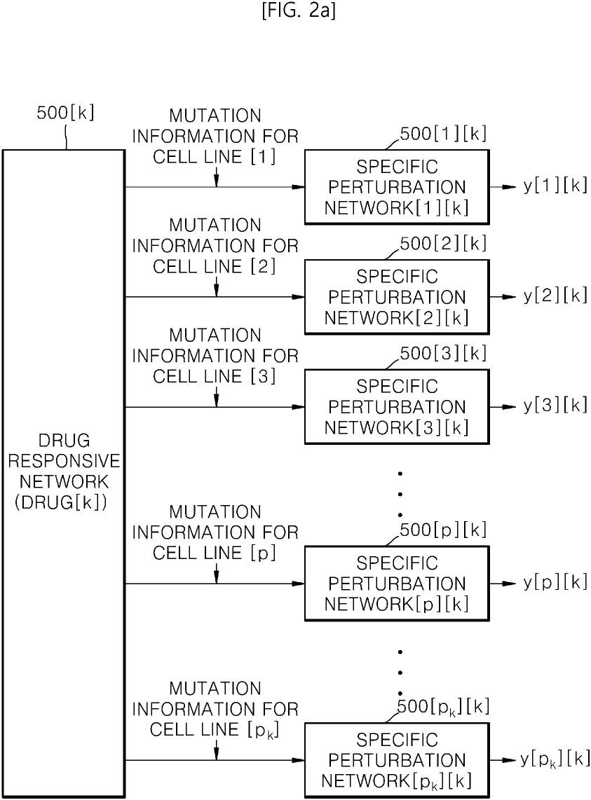

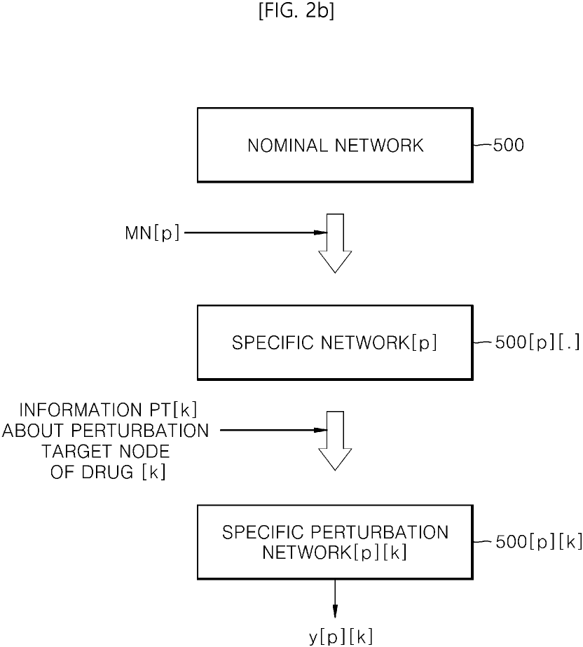

FIG. 2a shows a method of defining and generating a plurality of specific perturbation networks different from each other from a specific drug responsive network according to an embodiment of the present disclosure.

On the left side of FIG. 2a, a k-th drug responsive network 500[k], which is a drug responsive network for a drug [k], is presented. When pieces of mutation information for pₖ different cell lines are applied to the k-th drug responsive network 500[k], pₖ different specific perturbation networks may be defined. For example, when a p-th piece of mutation information among the pieces of mutation information for a total of pₖ different cell lines prepared in advance is applied to the k-th drug responsive network 500[k], a specific perturbation network [p][k] may be generated.

The specific perturbation network [p][k] may output cell death probability prediction values y[p][k] of the cancer cell line [p] when the drug [k] is administered to the cancer cell line [p],

The pₖ different cell lines may be selected from among the P cell lines that are in a population (P>pₖ). In addition, the pieces of mutation information for the pₖ different cell lines may be selected from mutation information for P cell lines.

In this case, information on the responsiveness to the drug [k] may not exist for all of the P cell lines. For example, the test of administering the drug [k] may be performed for some of the P cell lines, whereas the test of administering the drug [k] may not be performed for the other cell lines. That is, information on the responsiveness to the drug [k] may exist only for some of the P cell lines.

The pₖ different cell lines used to generate the specific perturbation networks from the k-th drug responsive network 500[k] may be composed of some of the P cell lines for which information on the responsiveness to the drug [k] exists.

The number of specific perturbation networks obtainable from each of different drug responsive networks may be different. For example, when the number of specific perturbation networks obtainable from the first drug responsive network for drug [1] is p₁, and the number of specific perturbation networks obtainable from the second drug responsive network for drug [2] is p₂, p₁ may be different from p₂.

In the specific perturbation network [p][k], state transition equations for determining the state value of each node over time may already be defined.

For example, the state transition equations for the specific perturbation network [p][k] may be basically the same as the state transition equations of the k-th drug responsive network 500[k]. However, for example, only one or a plurality of state transition equations for determining the state of a node corresponding to a position of a mutation existing in the specific perturbation network [p][k] may be modified.

FIG. 2b illustrates the method of generating the specific perturbation networks of FIG. 2a in another manner.

The specific network [p] may be generated by applying information MN[p] about a mutation-generating node of the cancer cell line [p] to the nominal network 500.

The specific perturbation network [p][k] may be generated by applying information PT[k] about a perturbation target node of the drug [k] to the generated specific network [p],

In this case, the specific perturbation network [p][k] may output cell death probability prediction values y[p][k] of the cancer cell line [p] when the drug [k] is administered to the cancer cell line [p].

The above-described FIGS. 2a and 2b may be collectively referred to as FIG. 2.

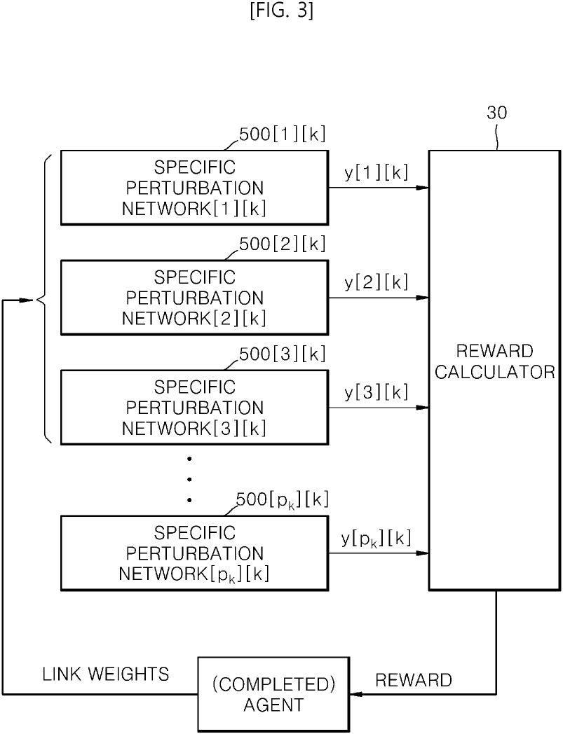

FIG. 3 shows a method of determining weights assigned to links of a specific drug responsive network according to an embodiment of the present disclosure.

FIG. 3 shows a framework for determining weights of the links of the k-th drug responsive network 500[k] related to the drug [k]. For the framework, a set of specific perturbation networks [p][k] generated from the k-th drug responsive network 500[k] described in FIG. 2 may be used (p = 1, 2, 3, ..., pₖ). In addition, the framework may use a reward calculator 30 and an agent 20 together.

In the present specification, the agent 20 may be referred to as a weight determination agent.

The agent 20 may be an information processing module including a network including a neural network. The agent 20 may include a learnable network such as a machine learning network or a neural network, and may include a plurality of layers. The neural network may be trained by reinforcement learning. The agent 20 used in FIG. 3 may have already been completed. A specific method for training the agent 20 will be described later in the present specification.

From the set of specific perturbation networks [p][k] presented in FIG. 3, the cell death probability prediction values y[p][k] of a set of cancer cell lines [p] may be output (p = 1, 2, 3, ..., pₖ).

Hereinafter, the y[p][k] output for the drug [k] may be simply expressed as yₚ, and an index pₖ may be represented by being replaced with an index N.

Now, a prediction vector Y={y₁, y₂, y₃, ..., yrr} may be generated by using pₖ cell death probability prediction values yₚ, that is, N cell death probability prediction values yₚ.

Furthermore, the result of observation through the in vitro test performed on an actual death rate of the cancer cell line [p] when the drug [k] is administered to the cancer cell line [p] may be prepared. The results performed through the in vitro test may be obtained from existing public data. Therefore, when the drug [k] is administered to the cancer cell line [p], the observation value zₚ regarding the actual death rate of the cancer cell line [p] may also be N from p = 1 to p = N (N = pₖ). An observation value vector Z={z₁, z₂, z₃, ..., z_{N}} may be generated using N observation values zₚ.

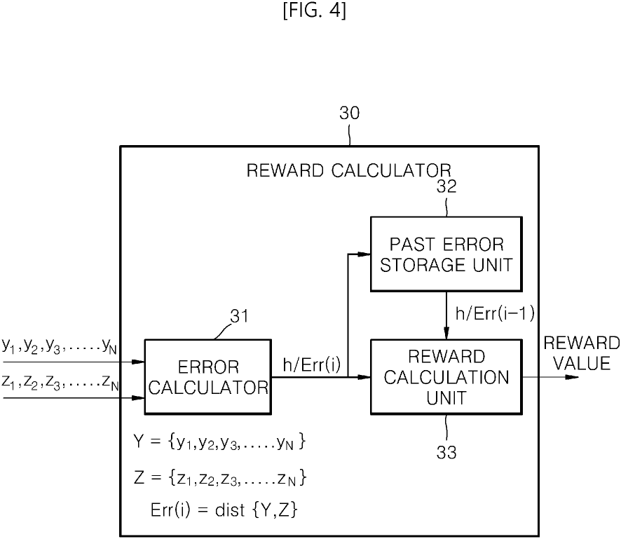

FIG. 4 is a block diagram illustrating a function of the reward calculator calculating a reward value by using the cell death probability prediction value yₚ calculated for the cancer cell line [p] and the observation value zₚ regarding the actual death rate of cancer cell line [p] when the drug [k] is administered to cancer cell line [p] in order to execute one learning step according to an embodiment of the present disclosure (p=1, 2, 3, ..., N).

The reward calculator 30 may calculate the reward values only when both the prediction values yₚ and the observation values zₚ for all values from p=1 to p=N are input.

In the present specification, the ‘prediction value’ may be referred to as a ‘simulation prediction value’, and the ‘observation value’ may also be referred to as an ‘in vitro observation value’.

An error calculator 31 may calculate a distance between the prediction vector Y composed of the prediction values yₚ for all values from p = 1 to p = N and the observation value vectors Z composed of the observation values zₚ for all values from p = 1 to p = N, and regard the distance as a prediction error Err(i) of the specific perturbation network. Here, i is an index indicating the learning step performed for the i-th time (learning iteration). The error calculator 31 may output a first value h/Err(i) inversely proportional to the prediction error Err(i).

The first value h/Err(i) may be stored in a past error storage unit 32 and used later. That is, for example, the first value h/Err(i) stored in the past error storage unit 32 may be used in connection with the i + 1-th learning step (learning iteration) which is executed after the storage has been made.

Similarly, in the past error storage unit 32, a second value h/Err(i-1) inversely proportional to the prediction error Err(i-1) which is obtained in the learning step performed for the i-1-th time may be already stored.

A reward calculation unit 33 may calculate the reward value based on a difference value between the first value h/Err(i) and the second value h/Err(i-1).

A specific method of calculating the reward value is as follows.

The reward entering the agent 20 in the i + 1-th learning step as an input may be calculated as follows.

First, the errors Err(0) to Err(i-1) obtained from the 0-th learning step to the i-1-th learning step are stored.

In this case, among the values h/Err(0), h/Err(1), h/Err(2), ..., h/Err(i -1) inversely proportional to the errors obtained from the 0-th learning step to the i-th learning step, a maximum value may be selected.

Assuming that the maximum value is h/Err(j), the value of d(i) may be calculated as in Equation 1 below (j = 0, 1, 2, ..., or i - 1).

$$\text{d(i) = h/Err(i) - h/Err(j)}$$

Here, Err(i) is an error value obtained in the i-th learning step.

Here, when d(i) is negative, the reward may be determined as 0 (zero), and when d(i) is positive, the reward may be determined as d(i).

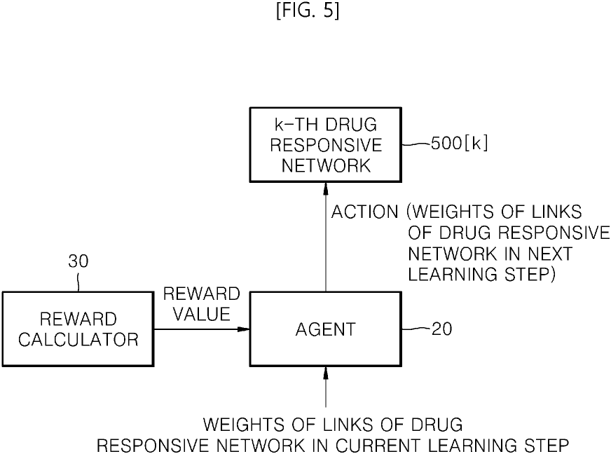

FIG. 5 is a block diagram illustrating a process of updating weights assigned to links of the nominal network using the calculated rewards.

In the process of one learning step, the reward calculator 30 outputs the reward value once. The outputted reward value is input to the agent 20. The agent 20 outputs an action based on the reward value. The action means a set of weights assigned to the links of the k-th drug responsive network 500[k] in the next learning step. By applying the output action, it is possible to update the weights assigned to each link of the k-th drug responsive network 500[k].

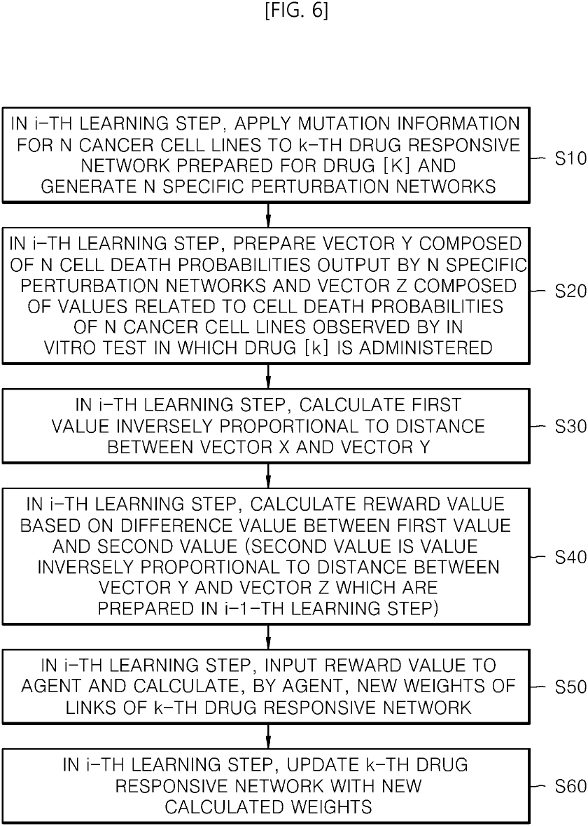

FIG. 6 is a flowchart showing a method of updating weights assigned to links in a drug responsive network related to a specific drug by one learning step, which is provided by an embodiment of the present disclosure.

The flowchart shown in FIG. 6 may be described with reference to FIGS. 2 to 5 together.

The method according to the flowchart of FIG. 6 may be executed by a computing device having a processing unit and a storage unit. The method may include, by the computing device, executing a predetermined learning step.

In the present specification, the learning step may be referred to as learning iteration.

The weights of the k-th drug responsive network 500[k] may be updated once by one learning step.

In this case, the learning step may include the following steps S10, S20, S30, S40, S50, and S60.

Step S10, step S20, step S30, step S40, step S50, and step S60 may be performed in the i-th learning step, and may be repeatedly executed for each different learning step.

In step S10, the computing device may apply the mutation information for N cancer cell lines to the k-th drug responsive network 500[k] prepared for the drug [k], and generate N specific perturbation networks [p][k] (p=1, 2, 3, ..., N(=pₖ)).

In step S20, the computing device may prepare the vector Y={yi, y₂, y₃, ..., y_{N}} composed of N cell death probabilities output by the N specific perturbation networks [p][k], and the vector Z={z₁, z₂, z₃, ..., z_{N}} composed of values related to the N percentage cell deaths of the N cancer cell lines observed by in vitro tests in which the drug [k] is administered.

In step S30, the computing device may calculate the first value h/Err(i) inversely proportional to the distance (dist{Y, Z}) between the vector Y and the vector Z.

In step S40, the computing device may calculate the reward value based on the difference value between the first value and a predetermined second value.

In this case, the second value may be the value h/Err(i-1) inversely proportional to the distance between the vector Y and the vector Z prepared in the i-1-th learning step performed immediately before the i-th learning step.

In step S50, the computing device may input the reward value to the agent 20, and the agent 20 may calculate new weights of the links of the k-th drug responsive network 500[k].

In step S60, the computing device may update the k-th drug responsive network 500[k] with the calculated new weights.

The computing device may be configured to repeatedly execute the learning step for a given k-th drug responsive network 500[k].

Whenever the learning step is repeated, the weights of the links of the k-th drug responsive network 500[k] may be updated once. That is, each time the learning iteration is performed once, the weights of the links of the k-th drug responsive network 500[k] may be updated once.

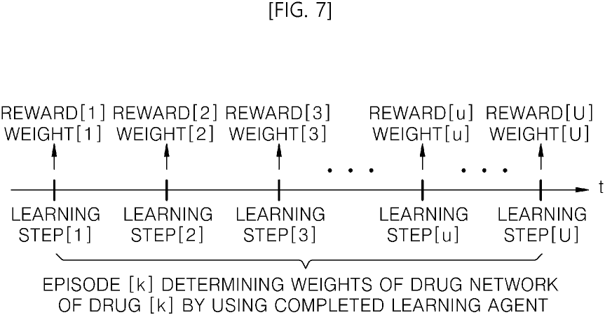

FIG. 7 shows a method of determining the weights of the drug responsive network as optimal values by using the weight update method of the drug responsive network described in FIG. 6.

A process in which the agent 20 receives an input once and performs an output related to the input may be referred to as one learning step.

With respect to the k-th drug responsive network 500[k] defined for the drug [k] that is a given drug, the learning step described in FIG. 6 may be repeatedly performed U times. In this case, in the process of executing the u-th learning step [u], the reward calculator 30 may output the reward [u], and the agent 20 may output the weight [u].

That is, by repeating the learning step U times, total U rewards may be generated. In this case, the best reward value may be selected from among the total U rewards. If a larger reward value is better, the largest reward value may be selected. The reward value selected in this way is the optimal reward value.

In this case, as the learning step is repeated, the reward value may not necessarily change to a better value. That is, as the learning step is repeated, the reward value may increase and then decrease again, or decrease and then increase again.

Next, the weights calculated in the learning step in which the optimal reward value is generated may be determined as the optimal weights.

The determined optimal weight may be finally determined as the weights of the links of the k-th drug responsive network 500[k].

### Deciding of K Different Drug Responsive Networks

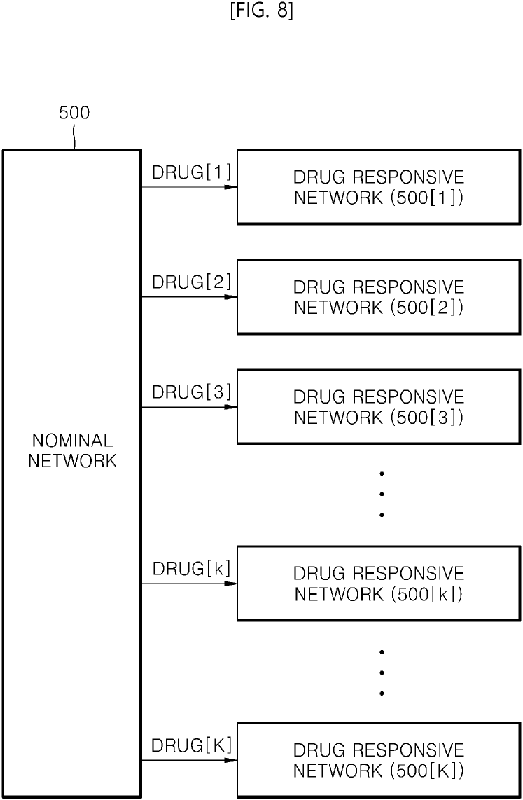

FIG. 8 illustrates the concept of deciding a plurality of different drug responsive networks from a given nominal network.

The contents described above in FIGS. 2 to 7 may be applied to one specific drug [k]. The techniques described in FIGS. 2 to 7 may be independently applied to each of the plurality of drugs. That is, the weights of K different drug responsive networks 500[k] defined for K different drugs may be decided by applying the techniques described in FIGS. 2 to 7.

That is, the structures of K different drug responsive networks 500[k] from one nominal network may be easily determined. However, the value of the weight assigned to each of the links of the K different drug responsive networks 500[k] may be decided by applying the techniques according to embodiments of the present disclosure described with reference to FIGS. 2 to 7.

### Process of Selecting Drug Suitable for Treatment of Specific Cancer Patient

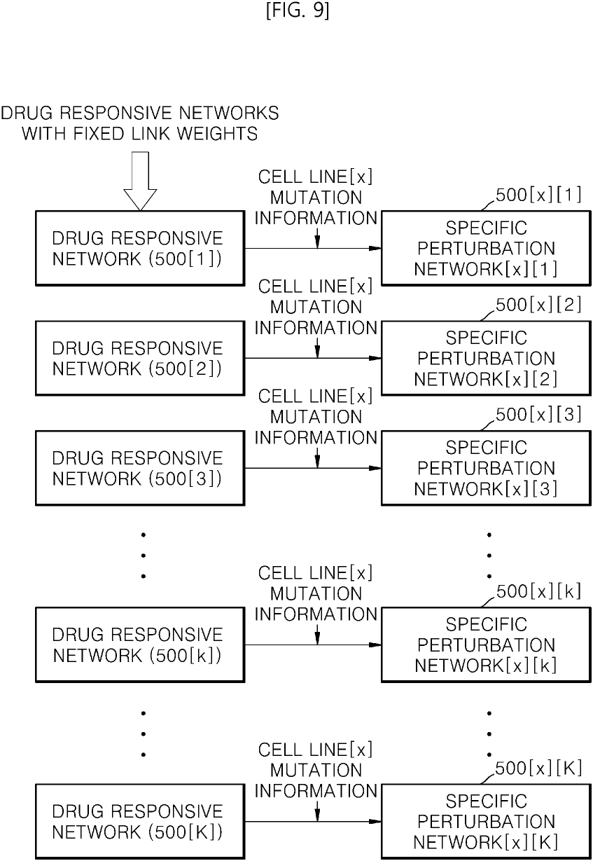

FIG. 9 is a diagram illustrating a process of finding a drug suitable for a patient [x] using a plurality of decided different drug responsive networks, according to an embodiment of the present disclosure.

Now, a situation may be assumed in which cancer treatment is required for a specific cancer patient, patient [x]. In addition, it is assumed that mutation information for a cell line [x], a cancer cell line of the patient [x], may be obtained. Further, it is assumed that a drug may be selected from a total of K drugs for the treatment of the patient [x]. The assumptions are fully feasible at the current technical level.

In addition, it is assumed that completed drug responsive networks for the K drugs are already prepared by the techniques of FIGS. 2 to 7 described above.

As shown in FIG. 9, by applying the mutation information for the cell line [x] to each of the K drug responsive networks 500[k], a total of K specific perturbation networks [x][k] may be generated (k=1, 2, 3, ..., K).

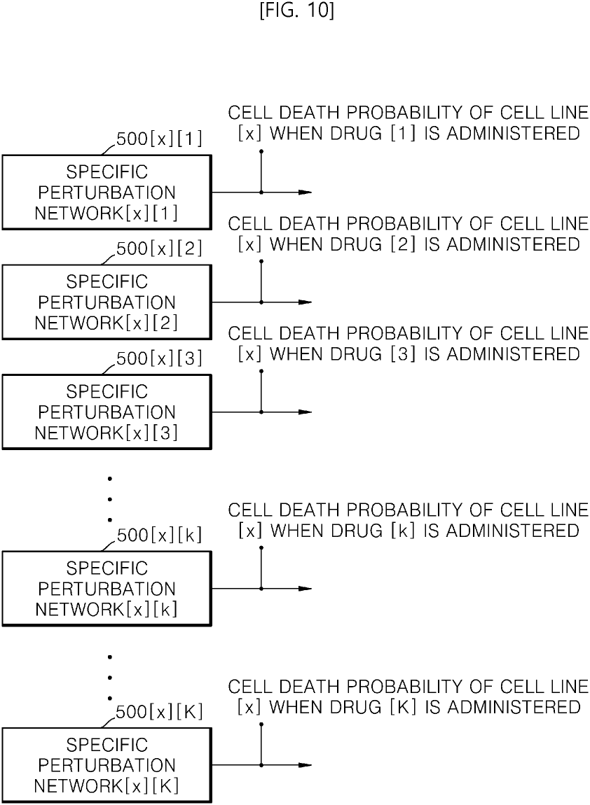

FIG. 10 shows a process of determining an optimal drug for a patient [k] using the K specific perturbation networks [x][k] prepared as in FIG. 9.

As shown in FIG. 10, each of the K specific perturbation networks [x][k] may output a simulation prediction value that predicts a cell death probability of the cell line [x] when the drug [k] is administered to the cell line [x].

Accordingly, a drug or drugs corresponding to the most desirable value among the K simulation prediction values may be proposed as a therapeutic agent for the patient [x]. The proposed therapeutic agent may be employed by a doctor or a new drug developer.

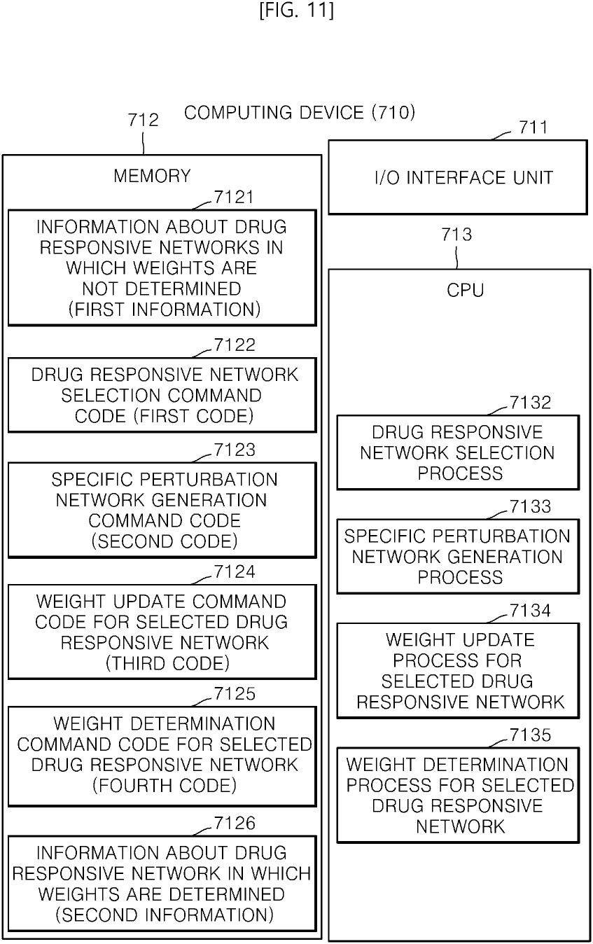

FIG. 11 shows a configuration of a computing device executing a method of completing a drug responsive network by determining weights of the drug responsive network according to an embodiment of the present disclosure.

A computing device 710 may include an input/output (I/O) interface unit 711, a memory 712, and a central processing unit (CPU) 713.

The memory 712 may store first information that is information for drug responsive networks in which the weights are not determined. The first information may include information 7121 about a state transition rule of the drug responsive networks.

In addition, the memory 712 may store a drug responsive network selection command code (simply, first code) 7122 configured to select one drug responsive network in which in-network weights are to be determined, from among drug responsive networks in which the weights are not determined.

Furthermore, the memory 712 may store a specific perturbation network generation command code (simply, second code) 7123 for generating N different specific perturbation networks by applying the mutation information for N different cancer cell lines to the selected drug responsive network.

Furthermore, the memory 712 may store a weight update command code (simply, third code) 7124 for the selected drug responsive network, which is configured to update the weights of the selected drug responsive network for each learning step using the method described in FIG. 3.

Furthermore, the memory 712 may store a weight determination command code (simply, fourth code) 7125 for the selected drug responsive network, which determines an optimal weight set among a plurality of weight sets output by the agent 20 for each of the plurality of times of the learning step.

Furthermore, the memory 712 may store second information 7126, which is information 7126 about the selected drug responsive network in which the weights are determined. The second information may include information about the state transition rule of the drug responsive networks and determined weight values.

The CPU 713 may read and use the first information 7121.

Further, the CPU 713 may read and execute the first to fourth codes 7122 to 7125.

The CPU 713 may use weight information generated by the fourth code 7125 to store, in the memory 712, information about a plurality of drug responsive networks in which the weights are determined.

In addition, the CPU 713 may execute the first code to execute a drug responsive network selection process of selecting one drug responsive network in which in-network weights are to be determined, from among drug responsive networks in which the weights are not determined. In this way, for example, the k-th drug responsive network 500[k] of FIG. 2a may be prepared.

The CPU 713 may execute the second code to execute a specific perturbation network generation process of generating N different specific perturbation networks by applying the mutation information for N different cancer cell lines to the selected drug responsive network. In this way, for example, the p-th drug responsive network 500[p][k] of FIG. 2a may be prepared (p=1, 2, 3, ....., pₖ(=N)).

Furthermore, the CPU 713 may execute the third code to update the weights of the selected drug responsive network for each learning step using the method described in FIG. 3, that is, execute a weight update process for the selected drug responsive network. This process may be carried out, for example, in the method described in FIG. 3.

Furthermore, the CPU 713 may execute the fourth code to determine an optimal weight set among a plurality of weight sets output by the agent 20 for each of the plurality of times of the learning step, that is, execute a weight determination process for the selected drug responsive network. This process may be carried out by using, for example, the results of a plurality of times of the learning step, which are performed in the process of executing the episode [k] shown in FIG. 7.

The CPU 713 uses the I/O interface unit 711 to provide information about the selected drug responsive network in which the weights are determined to another computing device, or to provide it as information for execution of a subsequent process of the computing device 710.

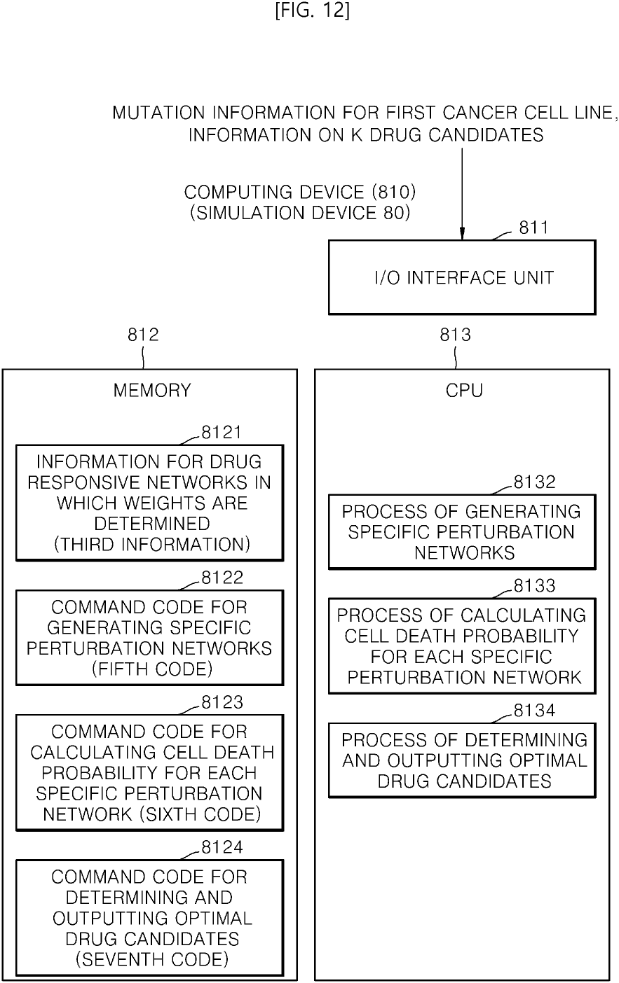

FIG. 12 shows the configuration of a computing device executing a simulation method for determining an optimal drug effective for killing a specific cancer cell line according to an embodiment of the present disclosure.

A computing device 810 may include an input/output (I/O) interface unit 811, a memory 812, and a central processing unit (CPU) 813.

The computing device 810 may receive information about K drug candidates and mutation information for the first cancer cell line through the I/O interface unit 811. The information about the K drug candidates may be information capable of specifying the K drugs. The first cancer cell line may be obtained from the body of a first patient, which is a specific patient.

The memory 812 may store third information 8121 that is information for drug responsive networks in which the weights are determined. The drug responsive networks in which the weights are determined may include K drug responsive networks generated from the K drugs. The third information 8121 may be the same as the second information 7126 stored in the memory 712 of FIG. 11. The third information may include drug responsive networks for more than k drugs.

In addition, the memory 812 may store a command code (simply, fifth code) 8122 for generating K specific perturbation networks by applying the mutation information for the first cancer cell line to each of the K drug responsive networks in which the weights are determined.

Furthermore, the memory 812 may store a command code (simply, sixth code) 8123 for calculating the cell death probability obtainable from each of the K specific perturbation networks. In this case, the cell death probability obtainable from the kth specific perturbation network among the K specific perturbation networks may be a simulation value representing the cell death probability of the first cancer cell line when the drug [k] is administered to the first cancer cell line.

In addition, the memory 812 may store a command code (simply, seventh code) 8124 for determining M drugs corresponding to M cell death probabilities selected from among the K cell death probabilities obtained from the K specific perturbation networks and including the determined drugs in optimal drug candidates, and outputting the optimal drug candidates (M <= K). The output of the optimal drug candidates may be executed through the I/O interface unit 811.

In a preferred embodiment, a drug corresponding to the highest cell death probability among the K cell death probabilities may be included in the optimal drug candidates.

The CPU 813 may read and use the third information 8121 that is information 7125 about the selected drug responsive network in which the weights of the links are determined. The third information may include, for example, information about the drug responsive network 500[k] shown in FIG. 9 (k=1, 2, 3, ..., K).

Further, the CPU 813 may read and execute the fifth to seventh codes 8122 to 8124.

The CPU 813 may execute the fifth code to execute the specific perturbation network generation process of generating K specific perturbation networks by applying the mutation information for the first cancer cell line to each of the K drug responsive networks in which weights are determined. Accordingly, for example, information about the specific perturbation network 500[x][k]shown in FIG. 9 may be included (k=1, 2, 3, ..., K, and x is an index indicating the first cancer cell line).

The CPU 813 executes the sixth code to execute, for each specific perturbation network, a cell death probability calculation process of calculating the cell death probability obtainable from each of the K specific perturbation networks. Thus, for example, when the drug [k] shown in FIG. 10 is administered to the first cancer cell line, a simulation value of the cell death probability of the first cancer cell line may be obtained (the first cancer cell line corresponds to the cell line [x]).

In addition, the CPU 813 may execute the sixth code to execute an optimal drug-candidates determination and output process of determining M drugs corresponding to M cell death probabilities selected from among the K cell death probabilities obtained from the K specific perturbation networks and including the determined drugs in the optimal drug candidates, and outputting the optimal drug candidates.

The CPU 813 uses the I/O interface unit 811 to provide information about the determined optimal drug candidates to another computing device, or to provide it as information for execution of a subsequent process of the computing device 810.

The computing device 710 of FIG. 11 and the computing device 810 of FIG. 12 may be provided independently from each other, or may be provided as one integrated device.

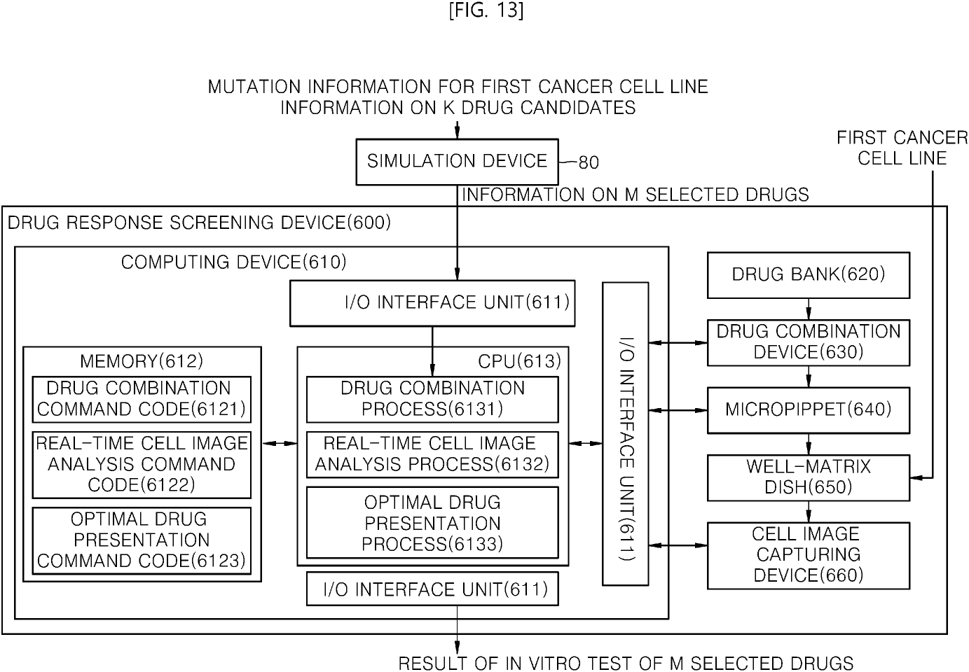

FIG. 13 shows a structure of a cancer treatment candidate drug determination system provided according to an embodiment of the present disclosure.

The cancer treatment candidate drug determination system may include a drug response screening device 600 and a simulation device 80 (computing device 810). The cancer treatment candidate drug determination system may further include the computing device 710 of FIG. 12.

The drug response screening device 600 may include a computing device 610, a drug bank 620, a drug combination device 630, a micropipette 640, a well-matrix dish 650, and a cell image capturing device 660.

The computing device 610 may include an input/output (I/O) interface unit 611, a memory 612, and a CPU 613. The memory 612 may store a drug combination command code 6121, a real-time cell image analysis command code 6122, and an optimal drug presentation command code 6123. The CPU 613 may read the drug combination command code 6121, the real-time cell image analysis command code 6122, and the optimal drug presentation command code 6123 to execute corresponding processes, that is, execute a drug combination process 6131, a real-time cell image analysis process 6132, and an optimal drug presentation process 6133, respectively.

The simulation device 80 of FIG. 13 may be the computing device 810 of FIG. 12 or an integrated device that integrates the computing device 710 of FIG. 11 and the computing device 810 of FIG. 12.

As described in FIG. 11, the simulation device 80 may receive the mutation information for the first cancer cell line and the information about K drug candidates, and provide information on the M selected drugs to the drug response screening device 600.

The I/O interface unit 611 may transmit the information on the M selected drugs to the CPU 613. The drug combination command process 6131 executed in the CPU 613 may transmit, to the drug combination device 630 and the micropipette 640, a command to extract the M selected drugs from the drug bank 620 using the information on the M selected drugs, and inject the extracted drugs into the well-matrix dish 650. The command may be transmitted through the I/O interface unit 611.

The drug bank 620 may be a drug reservoir in which a plurality of drugs including at least the M selected drugs are prepared.

Alternatively, the drug bank 620 may be a drug reservoir in which a plurality of drugs including at least the K drug candidates are prepared.

The drug combination device 630 may be a mechanical device that extracts a plurality of drugs stored in the drug bank 620 and provides the extracted drugs to the micropipette 640.

When any one of the M selected drugs is a first drug as a single drug, the drug combination device 630 may extract the first drug from the drug bank 620 and provide the extracted first drug to the micropipette 640.

When any one of the M selected drugs is a combination drug of the first drug and a second drug, the drug combination device 630 may extract the first drug and the second drug from the drug bank 620 and provide a combination drug combined with each other to the micropipette 640.

The well-matrix dish 650 may be a dish in which a plurality of wells are formed.

The drug response screening device 600 may be configured to inject culture solution of the first cancer cell line into M wells of the well-matrix dish 650 and store them.

The micropipette 640 may inject the drug or drug combination provided from the drug combination device 630 into one of the plurality of wells formed in the well-matrix dish 650.

The M selected drugs may be injected into M wells in which the culture solutions of the first cancer cell line are stored.

The cell survival rate and cell death rate of the first cancer cell line stored in the M wells may be determined with the administered drug.

The real-time cell image analysis command process 6132 may instruct the cell image capturing device 660 to capture images of the first cancer cell line in the M wells and return the resulting image to the real-time cell image analysis command process 6132, through the I/O interface unit 611.

The optimal drug presentation command process 6133 may generate information such as a value relating to the extent of cell growth and a value relating to the extent of cell death, in each of the M wells, that is, a cell death rate and a cell growth rate, and an area of a cell region in the well, based on the images transmitted by the cell image capturing device 660. In addition, information on drugs determined to be effective in killing cancer cells as the result of the in vitro test among the M selected drugs based on the generated information may be output by using a display screen, a speaker, a printer, or the like. Alternatively, the drug response screening device 600 may output the result of the in vitro test of each of the M selected drugs.

In an embodiment, all of the K drug candidates may be drugs approved for administration to actual patients. For example, all of the drugs included in the K drug candidates or drug bank may be FDA-approved drugs that may be prescribed directly to the patient. In this case, the information output by the drug response screening device 600 may be considered as a final candidate of a drug for treating a cancer patient of the first cancer cell line. In this case, the result of the in vitro test of the M selected drugs output by the interface unit 611 may be treated as useful information to a doctor who examines and treats a patient.

In another embodiment, all of the K drug candidates may be a single drug or a combination drug having an effect on cancer among candidate substances to be developed as drugs. That is, the drugs included in the K drug candidates or drug bank may be new drug candidate substances under development. Furthermore, the drugs included in the K drug candidates or drug bank may be those that have not yet been approved by the FDA. In this case, the information output by the drug response screening device 600 may not be used as information for treating a cancer patient of the first cancer cell line. However, the result of in vitro test of the M selected drugs output by the interface unit 611 may be treated as useful information for a new drug developer who develops a new drug.

As described above, by changing compositions constituting the drug bank included in the drug response screening device provided according to an embodiment of the present disclosure, a technology that is directly utilized in the field of patient treatment may be provided, or a technology that may be directly utilized in the field of new drug development may be provided.

At least one of the computing device 710 of FIG. 11, the computing device 810 of FIG. 12, and the computing device 610 of FIG. 13 may be provided as a single integrated device.

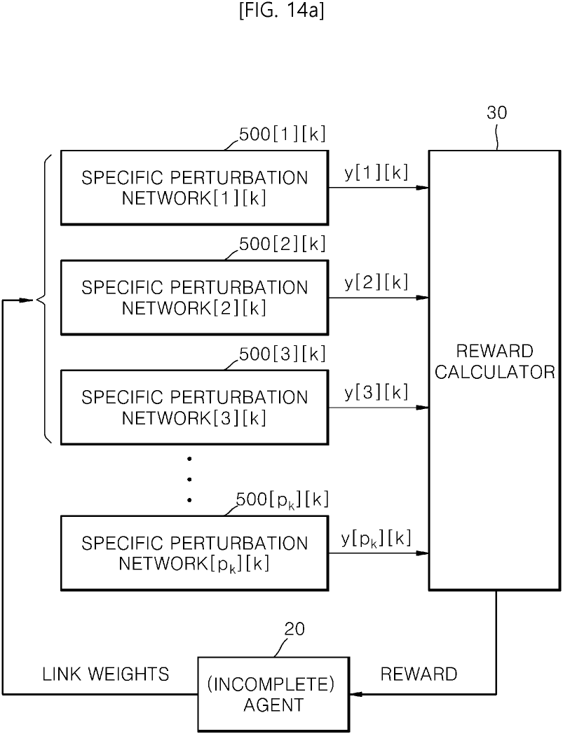

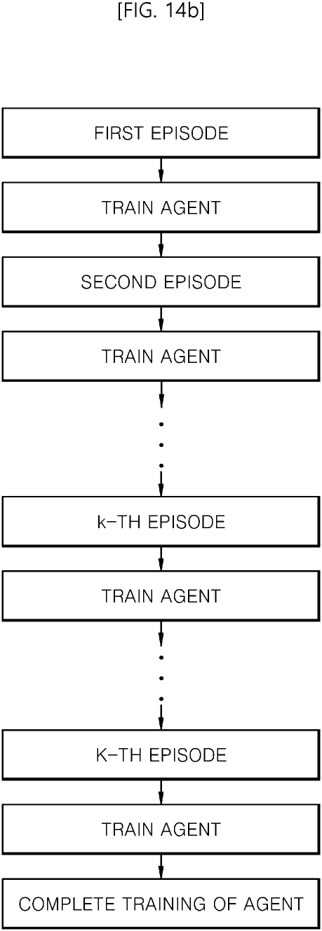

Hereinafter, FIGS. 14a and 14b, which will be described later, may be collectively referred to as FIG. 14.

FIG. 14 is a framework for describing a method of training the completed agent shown in FIG. 3.

The agent 20 may serve to determine weights assigned to links of the specific perturbation network composed of the links and nodes. When the weights assigned to the links of the specific perturbation network are determined as appropriate values, the specific perturbation network may more accurately output the cell death probability of the cell line.

The structure of the agent 20 may be designed in advance, but the input/output characteristics of the agent 20 or values of parameters assigned to the inside of the agent 20 for the operation of the agent 20 have to be updated from predetermined initial values to optimal values. For the update, the agent 20 has to be trained.

For training of the agent 20, some or all of a plurality of cancer cell lines for training, and a plurality of drugs (drug combination) may be used as training data.

A process in which the agent 20 receives an input once and performs an output related to the input may be referred to as one learning step.

For the one learning step, information about a set of cancer cell lines for training and one drug may be used. When the one drug is administered to each of the set of cancer cell lines for training in the in vitro test using an in vitro test device 90, the cell death probability of the set of cancer cell lines for training may be observed and determined. The percentage cell death may be presented, for example, as a vector Z composed of N scalar values.

Then, a set of specific perturbation networks may be generated by perturbing a set of specific networks obtained by modeling each of the set of cancer cell lines for training. In this case, the perturbed node in each specific network is a node corresponding to the protein on which the selected drug acts.

Further, a set of cell death probabilities that are obtainable from the set of specific perturbation networks may be calculated. The percentage cell death may be presented, for example, as a vector Y composed of N scalar values.

The agent 20 may receive the reward and/or the weights assigned to the links of the drug responsive network as input data.

The agent 20 may output updated information for weights to be assigned to the links of the drug responsive network in the next learning step based on the data input to the agent 20.

A set of a plurality of continuously executed learning steps may be referred to as a learning episode.

In an embodiment, for all learning steps in one episode, the drug used as training data may be limited to one. The drug for training may be changed after the episode has changed. However, the configuration of a first set of cancer cell lines for training used in the first learning step in one episode and a second set of cancer cell lines for training used in the second learning step in the one episode may be different from each other.

For one learning step, the values of the weights of the nominal network may be updated once. The agent 20 may be trained for each episode including a plurality of times of the learning step. Each time the episode is repeated, the amount of training of the agent 20 increases.

In an embodiment of the present disclosure, a total of K episodes may be executed to train the agent 20. In the present specification, an ‘episode’ refers to a unit for training the agent 20 once. That is, when the episode is executed a total of K times, the agent 20 is trained K times. In an embodiment of the present disclosure, one episode is associated with only one drug.

One episode may be executed by executing the learning step mentioned in FIG. 7 a plurality of times. Hereinafter, this will be described in detail.

FIG. 14a shows a framework of a k-th episode among K episodes for training the incomplete agent 20.

A structure shown in FIG. 14a is the same as the structure shown in FIG. 3. However, the agent 20 of FIG. 3 has a total of K training completed, whereas the agent shown in FIG. 14a is different in that a total of K training has not yet been completed.

FIG. 14b shows a process of completing the training of the agent by executing a plurality of times of episodes according to an embodiment of the present disclosure.

When the execution of the episode is finished once, the agent 20 may be trained once.

The k-th episode may include steps as follows (k=1, 2, 3, ..., K).

First, it is possible to select pₖ cell lines that are prepared through the in-vitro test by information on response to the drug [k], and prepare mutation information for the pₖ cell lines.

Second, pₖ specific perturbation networks may be generated by applying the mutation information for the pₖ prepared cell lines to the drug responsive network for the drug [k].

Third, the framework of FIG. 14a may be built using the pₖ generated specific perturbation networks.

Fourth, it is possible to execute the learning step Uₖ times using the framework of FIG. 14a built above.

When the k-th episode is completed, the agent 20 may be trained once by using Uₖ sets of link weights output by the agent 20 and Uₖ rewards input to the agent 20 in the process of executing the learning step Uₖ times.

In an embodiment, when k1 and k2 are different, pₖ₁ and pₖ₂ may be different, and Uₖ₁ and Uₖ₂ may be different.

Since the agent 20 is trained through the process of determining the weights of different K drug responsive networks, it may not be used only to determine the weights of a drug responsive network for a specific drug.

### Method of Obtaining Percentage Cell Death Z by Administering Specific Drug to Cancer Cell Lines

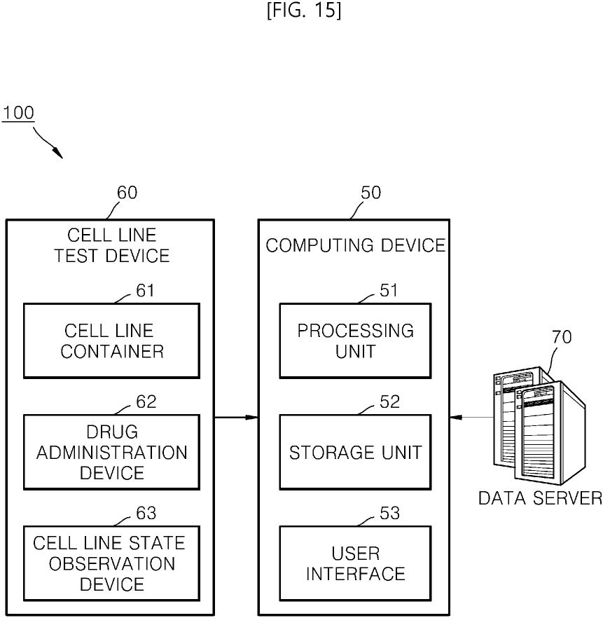

FIG. 15 shows a configuration of a system for obtaining and providing a percentage cell death Z by administering a specific drug to cancer cell lines, according to an embodiment of the present disclosure.

A biological network generation system 100 may include a computing device 50, a cell line test device 60, and a data server 70.

The cell line test device 60 may include a cell line container 61, a drug administration device 62, and a cell line state observation device 63.

In a plurality of wells provided in the cell line container 61, for example, cancer cell lines may be separately provided.

The drug administration device 62 may administer a selected specific drug to the cancer cell lines provided in the cell line container 61.

The cell line state observation device 63 may observe and output the percentage cell death of the cancer cell lines after the specific drug is administered.

The cell line test device 60 may be configured to provide the observed percentage cell deaths to the computing device 50.

The data server 70 may provide the computing device 50 with information about the drug responsive network for the specific drug. The information about the drug responsive network may include a configuration regarding the interconnection structure of nodes and links of a sub-network portion responding to the drug in the nominal network of the cancer cell lines. In addition, when the specific drug is administered to the cancer cell lines, the data server 70 may provide the computing device 50 with information on nodes affected by the specific drug.

The computing device 50 may include a processing unit 51, a storage unit 52, and a user interface 53.

The user interface 53 may receive information indicating the specific drug and information indicating the cancer cell lines from the user.

The computing device 50 may transmit the input information indicating the cancer cell lines and information indicating the specific drug to the cell line test device 60, and request, from the cell line test device 60, observation values for the percentage cell deaths of the cancer cell lines after the specific drug is administered to the cancer cell lines. The observed percentage cell deaths obtained from the cell line test device 60 may be stored in the storage unit 52 of the computing device 50.

The processing unit 51 is configured to execute a step of performing the learning step a plurality of times for determining the weights of the drug responsive network of the specific drug. The example has been described in FIGS. 3 to 7.

### Operating Principle of Agent

Hereinafter, an operating principle of the agent 20 will be described.

In the current learning step, the current weights, which are weights assigned to the links of the network 500, 520, or 521, and node characteristic values are input to a graph neural network, and then the values and the reward value obtained for the current weights are used as input of a recurrent neural network (RNN). The RNN may output actions by putting together the input values in the current learning step (weights, reward) and the hidden state containing information of the previous learning step. The actions may be update weights used in the next learning step. The update weights are weights assigned to the links of the network 500, 520, or 521 in the next learning step.

The agent 20 may include three portions of an input layer, a submodule layer, and a main layer.

The input layer may include a graph module for embedding a graph and a message module for inter-agent communication. There are two graph neural networks of the same structure in the graph module; one (G) is a module for a global state estimator and a main layer, and the other (G^{c}) is a module for a context estimator. A message module G^{m} may be used in all modules of the submodule layer and the main layer. Any module in the layers may have a graph neural network structure. The graph module may receive node features (8 centrality measures) and link features (weights, edge betweenness centrality) for every learning step and output nodes, links, and global features. The message module may receive 0 (zero) vector input at the start of each episode, and may recursively receive the previous calculation value in the subsequent learning step. Based on each link, the output of three modules may be reconstructed by concatenating source node features, target nodes, link features, and a global state, and may be used as the state to be input to a subsequent module. A state of the i-th link reconstructed in each of the three modules (G, G^{c}, G^{m}) is expressed as in Equation 2.

$$\begin{matrix}{\text{L}}_{\text{i}} \text{=} \left(\text{ni\_s, ni\_t, li, g}\right) \text{,} & \\ {\text{L}}_{\text{i}} {}^{\text{C}} \text{=} \left({\text{ni\_s}}^{\text{C}} \text{,} {\text{ni\_t}}^{\text{C}} \text{,} {\text{li}}^{\text{C}} {\text{, g}}^{\text{C}}\right) & \\ {\text{L}}_{\text{i}} {}^{\text{m}} \text{=} \left({\text{ni\_s}}^{\text{m}} \text{,} {\text{ni\_t}}^{\text{m}} \text{,} {\text{li}}^{\text{m}} {\text{, g}}^{\text{m}}\right) & \end{matrix} ,$$

The submodules and modules of the main layer may receive the reconstructed state as an input.

The submodule layer may include the context estimator module and a global state estimator module. The two modules are 1-layer LSTMs and share weights for individual coordinate inputs, but may maintain independent hidden states. The context estimator is a module for estimating information about the environment, and may input L^{c}, L^{m}, and the reward value of the previous learning step into the LSTM, and then output the information about the environment through two dense layers to which elu activation is applied. The global state estimator is a module for learning the agent-to-agent communication protocol, and may receive L, L^{m}, and the action of the current learning step, and output the state of the next learning step (L of the next learning step).

In the main layer, L, L^{m}, the reward value of the previous learning step, and the outputs of two submodules may be input to the 2-layer LSTM, and the action may be outputted through one dense layer to which elu activation is applied.

### Business Model Using the Present Disclosure

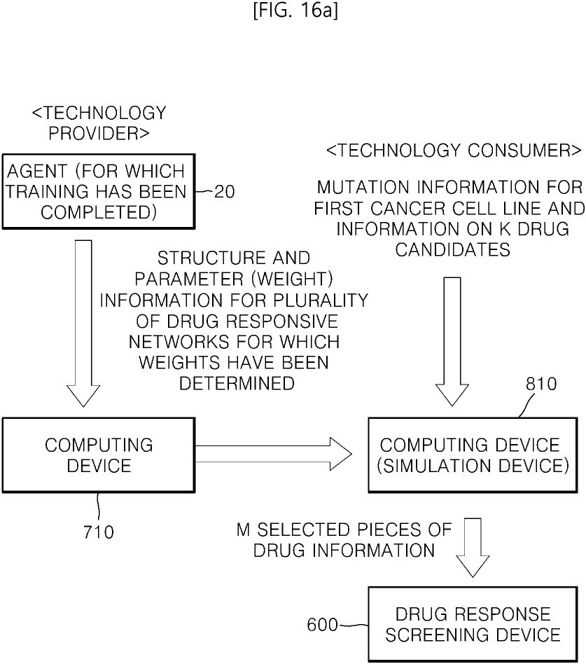

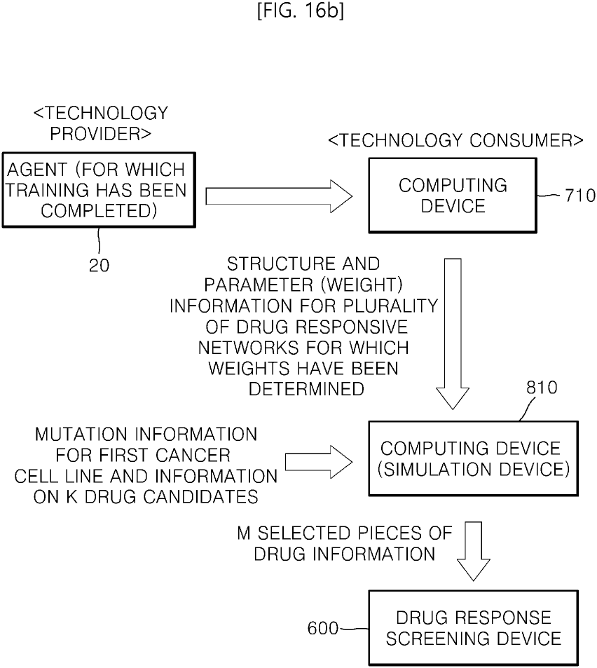

FIG. 16a shows one example of a business model using the present disclosure.

A technology provider may provide the computing device 710 with the agent 20 for which training has been completed with the technology presented in FIG. 14. In addition, the computing device 710 may output structure and parameter (weight) information for a plurality of drug responsive networks in which the weights are determined. This information may be transmitted to a technology consumer. The task of training the agent may also be executed in the computing device 710.

The technology consumer may input, to the computing device 810, structure and parameter (weight) information for the plurality of drug responsive networks for which the weights have been determined, and mutation information for the first cancer cell line and information on K drug candidates (see FIG. 13). The computing device 810 may provide the M selected pieces of drug information to the drug response screening device 600. The drug response screening device 600 may output the result of the in vitro test of M selected drugs required by medical personnel or new drug developers.

FIG. 16b shows another example of a business model using the present disclosure.

The technology provider may provide the technology consumer with the agent 20 for which training has been completed with the technology presented in FIG. 14.

The technology consumer may install the provided agent 20 in the computing device 710. The computing device 710 may output the structure and parameter (weight) information for the plurality of drug responsive networks in which the weights are determined. To the computing device 810, the input structure and parameter (weight) information for the plurality of drug responsive networks for which the weights have been determined may be input, and mutation information for the first cancer cell line and the information on K drug candidates may be input (see FIG. 13). The computing device 810 may provide the M selected pieces of drug information to the drug response screening device 600. The drug response screening device 600 may output the result of the in vitro test of M selected drugs required by medical personnel or new drug developers.

In FIGS. 16a and 16b, the results of in vitro tests of the M selected drugs may be utilized by a doctor. In this case, the K drugs corresponding to the information on K drug candidates input to the computing device 810 are FDA-approved drugs, and may be a commercially available drug set that a doctor may use immediately.

In contrast, the result of the in vitro test of the M selected drugs may be utilized by new drug developers. The K drugs corresponding to the information on K drug candidates input to the computing device 810 may be a set of substances having drug effects in a stage prior to being developed into drugs.

According to the existing technology, the weights of the cancer cell biological network, which is defined to find the optimal drug to be administered to a specific cancer patient are determined considering the type of cancer of the cancer patient and the location of the mutation specifically occurring in the cancer patient together. Therefore, the cancer cell biological network for each cancer patient has to be individually defined.

However, according to the present disclosure, a technique can be provided for determining weights associated with links of a cancer cell biological network that may be commonly applied to various types of cancer and various patients regardless of the type of cancer and the location of a mutation.

According to the present disclosure, a technique can be provided for optimizing parameters of a modeled biological network through machine learning to give the biological meaning of an internal structure thereof. Therefore, it is possible to not only select the optimal drug for cancer treatment using machine learning, but also generate data suitable for interpreting the biological significance suggested by parameters decided through machine learning.

According to the present disclosure, a technique can be provided for for training an agent (a weight determining agent) that plays a role in determining weights assigned to the links in a biological network composed of nodes and links. In addition, the present disclosure is to provide a technique for selecting a drug suitable for the treatment of a new cancer patient using the agent for which training has been completed.

By using the embodiments of the present disclosure described above, those skilled in the technical field to which the present disclosure belongs could easily implement various changes and modifications without departing from the scope of the essential characteristics of the present disclosure. Features of each claim in Claims may be incorporated into other claims that do not depend on or are not depended on by the claim, within the scope that could be understood upon reading the present specification.

What is claimed is:

1. A method for determining a cancer treatment candidate drug, the method comprising:
generating, by a simulation device, a plurality of specific perturbation networks by applying mutation information for a first cancer cell line to each of a plurality of drug responsive networks for a plurality of drugs;
selecting, by the simulation device, a plurality of candidate drugs from among the plurality of drugs based on a plurality of cell death probabilities for the first cancer cell line output by the plurality of specific perturbation networks;
providing information on the plurality of determined candidate drugs to a drug response screening device;
performing, by the drug response screening device, an in vitro test in which the plurality of candidate drugs are administered to a plurality of wells in which the first cancer cell line is stored;
capturing, by the drug response screening device, images of the first cancer cell line in the plurality of wells using a cell image capturing device to analyze the captured images; and
outputting, by the drug response screening device, a result of an in vitro test for at least some of the plurality of candidate drugs based on the analysis result.

2. The method of claim 1, further comprising, prior to the generating, performing, by a computing device, a process (=episode) of determining weights of a k-th drug responsive network responding to a k-th drug among the plurality of drug responsive networks,
wherein in the performing of the process, an agent that has been trained by reinforcement learning is used, and
the performing of the process comprises:
obtaining, by the computing device, mutation information for N (=pₖ) cell lines in which information on responsiveness by the in vitro test using the k-th drug among the plurality of drugs exists;
generating, by the computing device, N specific perturbation networks by applying the N pieces of mutation information to the k-th drug responsive network responding to the k-th drug;
repeatedly performing, by the computing device, a learning step a plurality of times by using the agent, the learning step being provided for updating the weights of the links of the k-th drug responsive network;
observing, by the computing device, each reward provided to the agent at each learning step;
selecting, by the computing device, a learning step corresponding to a reward with a largest value among the rewards observed at the observing step; and
deciding, by the computing device, that the link weights output by the agent in the selected learning step are weights of the links of the k-th drug responsive network.

3. The method of claim 2, wherein the agent is configured to determine weights of the links of the k-th drug responsive network in a next learning step, based on the reward and the weights of the links of the k-th drug responsive network in a current learning step.

4. The method of claim 3, wherein a process of determining the reward comprises:
preparing, by the computing device, a vector Y composed of N cell death probabilities output by the N specific perturbation networks and a vector z composed of N values related to a percentage cell death of the first cancer cell line observed by the in vitro test in which the k-th drug is administered to the first cancer cell line, in the current learning step in the plurality of times of the learning step;
calculating, by the computing device, a first value inversely proportional to a distance between the vector Y and the vector Z; and
calculating, by the computing device, the reward based on a difference value between the first value and a second value, and
the second value is a value inversely proportional to a distance between the vector Y and the vector Z prepared in the learning step immediately before the current learning step.

5. The method of claim 2, further comprising training, by the computing device, the agent before the performing of the process (=episode) of determining the weights of the k-th drug responsive network,
wherein in the training of the agent, a process (=episode) of training the agent is repeatedly performed for different G drugs, and
the process of training the agent that is performed for a g-th drug comprises:
obtaining, by the computing device, p_{g} pieces of mutation information for cell lines in which information on responsiveness by the in vitro test using the g-th drug is present;
generating, by the computing device, p_{g} specific perturbation networks by applying the p_{g} pieces of mutation information to a p-th drug responsive network responding to a p-th drug;
repeatedly performing, by the computing device, a learning step a plurality of times by using the agent, the learning step being provided for updating the weights of the links of the g-th drug responsive network; and
training, by the computing device, the agent by using the rewards provided to the agent during the plurality of learning steps and the weights obtained in a process of repeatedly performing the learning step a plurality of times.

6. The method of claim 5, wherein the agent is configured to determine weights of the links of the g-th drug responsive network in the next learning step, based on the reward and the weights of the links of the g-th drug responsive network in the current learning step.

7. A method for determining a cancer treatment candidate drug, the method comprising:
performing, by a computing device, a process (=episode) of determining weights of a k-th drug responsive network responding to a k-th drug among a plurality of drug responsive networks for a plurality of drugs;
generating, by a simulation device, a plurality of specific perturbation networks by applying mutation information for a first cancer cell line to each of the plurality of drug responsive networks; and
selecting, by the simulation device, a plurality of candidate drugs from among the plurality of drugs based on a plurality of cell death probabilities for the first cancer cell line output by the plurality of specific perturbation networks,
wherein in the performing of the process, an agent that has been trained by reinforcement learning is used, and
the performing of the process comprises:
obtaining, by the computing device, mutation information for N (=pₖ) cell lines in which information on responsiveness by the in vitro test using the k-th drug among the plurality of drugs exists;
generating, by the computing device, N specific perturbation networks by applying the N pieces of mutation information to the k-th drug responsive network responding to the k-th drug;
repeatedly performing, by the computing device, a learning step a plurality of times by using the agent, the learning step being provided for updating the weights of the links of the k-th drug responsive network;
selecting, by the computing device, the learning step when a reward provided to the agent has a largest value among the plurality of times of the learning step; and
deciding, by the computing device, that the link weights output by the agent in the selected learning step are weights of the links of the k-th drug responsive network.

8. A system for determining a cancer treatment candidate drug, the system comprising:
a simulation device; and
a drug response screening device,
wherein the simulation device is configured to:
generate a plurality of specific perturbation networks by applying mutation information for a first cancer cell line to each of a plurality of drug responsive networks for a plurality of drugs;
select a plurality of candidate drugs from among the plurality of drugs based on a plurality of cell death probabilities for the first cancer cell line output by the plurality of specific perturbation networks; and
provide information on the plurality of determined candidate drugs to a drug response screening device, and
the drug response screening device is configured to:
perform an in vitro test in which the plurality of candidate drugs are administered to a plurality of wells in which the first cancer cell line is stored;
capture images of the first cancer cell line in the plurality of wells using a cell image capturing device to analyze the captured images; and
output a result of an in vitro test for at least some of the plurality of candidate drugs based on the analysis result.

9. The system of claim 8, further comprising a computing device,
wherein the computing device is configured to perform a process (=episode) of determining weights of a k-th drug responsive network responding to a k-th drug among the plurality of drug responsive networks before the simulation generates the plurality of specific perturbation networks,
in performing the process, an agent that has been trained by reinforcement learning is used, and
the performing of the process comprises:
obtaining, by the computing device, mutation information for N (=pₖ) cell lines in which information on responsiveness by the in vitro test using the k-th drug among the plurality of drugs exists;
generating, by the computing device, N specific perturbation networks by applying the N pieces of mutation information to the k-th drug responsive network responding to the k-th drug;
repeatedly performing, by the computing device, a learning step a plurality of times by using the agent, the learning step being provided for updating the weights of the links of the k-th drug responsive network;
selecting, by the computing device, the learning step when a reward provided to the agent has a largest value among the plurality of times of the learning step; and deciding, by the computing device, that the link weights output by the agent in the selected learning step are weights of the links of the k-th drug responsive network.

10. The system of claim 9, wherein the agent is configured to determine weights of the links of the k-th drug responsive network in a next learning step, based on the reward and the weights of the links of the k-th drug responsive network in a current learning step.

11. The system of claim 10, wherein a process of determining the reward comprises:
preparing, by the computing device, a vector Y composed of N cell death probabilities output by the N specific perturbation networks and a vector z composed of N values related to a percentage cell death of the first cancer cell line observed by the in vitro test in which the k-th drug is administered to the first cancer cell line, in the current learning step in the plurality of times of the learning step;
calculating, by the computing device, a first value inversely proportional to a distance between the vector Y and the vector Z; and
calculating, by the computing device, the reward based on a difference value between the first value and a second value, and
the second value is a value inversely proportional to a distance between the vector Y and the vector Z prepared in the learning step immediately before the current learning step.

12. The system of claim 9, wherein the computing device is configured to train the agent before performing the process (=episode) of determining the weights of the k-th drug responsive network,
in training the agent, a process (=episode) of training the agent is repeatedly performed for different G drugs, and
the process of training the agent that is performed for a g-th drug comprises:
obtaining, by the computing device, p_{g} pieces of mutation information for cell lines in which information on responsiveness by the in vitro test using the g-th drug is present;
generating, by the computing device, p_{g} specific perturbation networks by applying the p_{g} pieces of mutation information to a p-th drug responsive network responding to a p-th drug;
repeatedly performing, by the computing device, a learning step a plurality of times by using the agent, the learning step being provided for updating the weights of the links of the g-th drug responsive network; and
training, by the computing device, the agent by using reward values and the weights obtained in a process of repeatedly performing the learning step a plurality of times.

13. The system of claim 12, wherein the agent is configured to determine weights of the links of the g-th drug responsive network in the next learning step, based on the reward and the weights of the links of the g-th drug responsive network in a current learning step.

14. A system for determining a cancer treatment candidate drug, comprising:
a simulation device;
a drug response screening device; and
a computing device,
wherein the computing device is configured to perform a process (=episode) of determining weights of a k-th drug responsive network responding to a k-th drug among a plurality of drug responsive networks,
the simulation device is configured to:
generate a plurality of specific perturbation networks by applying mutation information for a first cancer cell line to each of a plurality of drug responsive networks for a plurality of drugs; and
select a plurality of candidate drugs from among the plurality of drugs based on a plurality of cell death probabilities for the first cancer cell line output by the plurality of specific perturbation networks,
in the performing of the process, an agent that has been trained by reinforcement learning is used, and
the performing of the process comprises:
obtaining, by the computing device, mutation information for N (=pₖ) cell lines in which information on responsiveness by the in vitro test using the k-th drug among the plurality of drugs exists;
generating, by the computing device, N specific perturbation networks by applying the N pieces of mutation information to the k-th drug responsive network responding to the k-th drug;
repeatedly performing, by the computing device, a learning step a plurality of times by using the agent, the learning step being provided for updating the weights of the links of the k-th drug responsive network;
selecting, by the computing device, the learning step when a reward provided to the agent has a largest value among the plurality of times of the learning step; and
deciding, by the computing device, that the link weights output by the agent in the selected learning step are weights of the links of the k-th drug responsive network.
